SEQUENCE LISTING (83 sequences)

SEQ ID NO: 1 - protein, 24 aa, Artificial Sequence
  Description of Artificial Sequence Synthetic peptide
ITDGEATDSGNIDAAKDIIYIIGI

SEQ ID NO: 2 - protein, 24 aa, Artificial Sequence
  Description of Artificial Sequence Synthetic peptide
GVDVDQDGETELIGAPLFYGEQRG

SEQ ID NO: 3 - protein, 25 aa, Artificial Sequence
  Description of Artificial Sequence Synthetic peptide
DLSYSLDDLRNVKKLGGDLLRALNE

SEQ ID NO: 4 - protein, 10 aa, Artificial Sequence
  Description of Artificial Sequence Synthetic peptide
ITDGEATDSG

SEQ ID NO: 5 - protein, 6 aa, Artificial Sequence
  Description of Artificial Sequence Synthetic peptide
ITDGEA

SEQ ID NO: 6 - protein, 6 aa, Artificial Sequence
  Description of Artificial Sequence Synthetic peptide
TDGEAT

SEQ ID NO: 7 - protein, 6 aa, Artificial Sequence
  Description of Artificial Sequence Synthetic peptide
DGEATD

SEQ ID NO: 8 - protein, 6 aa, Artificial Sequence
  Description of Artificial Sequence Synthetic peptide
GEATDS

SEQ ID NO: 9 - protein, 6 aa, Artificial Sequence
  Description of Artificial Sequence Synthetic peptide
EATDSG

SEQ ID NO: 10 - protein, 4 aa, Artificial Sequence
  Description of Artificial Sequence Synthetic peptide
DGEA

SEQ ID NO: 11 - protein, 10 aa, Artificial Sequence
  Description of Artificial Sequence Synthetic peptide
GVDVDQDGET

SEQ ID NO: 12 - protein, 10 aa, Artificial Sequence
  Description of Artificial Sequence Synthetic peptide
GETELIGAPL

SEQ ID NO: 13 - protein, 10 aa, Artificial Sequence
  Description of Artificial Sequence Synthetic peptide
APLYGEQRGK

SEQ ID NO: 14 - protein, 12 aa, Artificial Sequence
  Description of Artificial Sequence Synthetic peptide
XITDGEATDSGC

SEQ ID NO: 15 - protein, 8 aa, Artificial Sequence
  Description of Artificial Sequence Synthetic peptide
XITDGEAC

SEQ ID NO: 16 - protein, 8 aa, Artificial Sequence
  Description of Artificial Sequence Synthetic peptide
XTDGEATC

SEQ ID NO: 17 - protein, 8 aa, Artificial Sequence
  Description of Artificial Sequence Synthetic peptide
XDGEATDC

SEQ ID NO: 18 - protein, 8 aa, Artificial Sequence
  Description of Artificial Sequence Synthetic peptide
XGEATDSC

SEQ ID NO: 19 - protein, 8 aa, Artificial Sequence
  Description of Artificial Sequence Synthetic peptide
XEATDSGC

SEQ ID NO: 20 - protein, 6 aa, Artificial Sequence
  Description of Artificial Sequence Synthetic peptide
XDGEAC

SEQ ID NO: 21 - protein, 12 aa, Artificial Sequence
  Description of Artificial Sequence Synthetic peptide
XGVDVDQDGETC

SEQ ID NO: 22 - protein, 12 aa, Artificial Sequence
  Description of Artificial Sequence Synthetic peptide
XGETELIGAPLC

SEQ ID NO: 23 - protein, 12 aa, Artificial Sequence
  Description of Artificial Sequence Synthetic peptide
XAPLYGEQRGKC

SEQ ID NO: 24 - protein, 3 aa, Artificial Sequence
  Description of Artificial Sequence Synthetic peptide
ITD

SEQ ID NO: 25 - protein, 4 aa, Artificial Sequence
  Description of Artificial Sequence Synthetic peptide
ITDG

SEQ ID NO: 26 - protein, 21 aa, Artificial Sequence
  Description of Artificial Sequence Synthetic peptide
QTSVSPSKVILPRGGSVLVTG

SEQ ID NO: 27 - protein, 24 aa, Artificial Sequence
  Description of Artificial Sequence Synthetic peptide
DGPKLLGIETPLPKKELLPGNNRK

SEQ ID NO: 28 - protein, 10 aa, Artificial Sequence
  Description of Artificial Sequence Synthetic peptide
PSKVILPRGG

SEQ ID NO: 29 - protein, 10 aa, Artificial Sequence
  Description of Artificial Sequence Synthetic peptide
QTSVSPSKVI

SEQ ID NO: 30 - protein, 10 aa, Artificial Sequence
  Description of Artificial Sequence Synthetic peptide
LPRGGSVLVT

SEQ ID NO: 31 - protein, 10 aa, Artificial Sequence
  Description of Artificial Sequence Synthetic peptide
ETPLPKKELL

SEQ ID NO: 32 - protein, 10 aa, Artificial Sequence
  Description of Artificial Sequence Synthetic peptide
DQPKLLGIET

SEQ ID NO: 33 - protein, 10 aa, Artificial Sequence
  Description of Artificial Sequence Synthetic peptide
ELLLPGNNRK

SEQ ID NO: 34 - protein, 12 aa, Artificial Sequence
  Description of Artificial Sequence Synthetic peptide
XQTSVSPSKVIC

SEQ ID NO: 35 - protein, 12 aa, Artificial Sequence
  Description of Artificial Sequence Synthetic peptide
XLPRGGSVLVTC

SEQ ID NO: 36 - protein, 12 aa, Artificial Sequence
  Description of Artificial Sequence Synthetic peptide
XETPLPKKELLC

SEQ ID NO: 37 - protein, 12 aa, Artificial Sequence
  Description of Artificial Sequence Synthetic peptide
XDQPKLLGIETC

SEQ ID NO: 38 - protein, 12 aa, Artificial Sequence
  Description of Artificial Sequence Synthetic peptide
XELLLPGNNRKC

SEQ ID NO: 39 - protein, 6 aa, Artificial Sequence
  Description of Artificial Sequence Synthetic peptide
PKSVIL

SEQ ID NO: 40 - protein, 6 aa, Artificial Sequence
  Description of Artificial Sequence Synthetic peptide
SKVILP

SEQ ID NO: 41 - protein, 6 aa, Artificial Sequence
  Description of Artificial Sequence Synthetic peptide
KVILPR

SEQ ID NO: 42 - protein, 6 aa, Artificial Sequence
  Description of Artificial Sequence Synthetic peptide
VILPRG

SEQ ID NO: 43 - protein, 6 aa, Artificial Sequence
  Description of Artificial Sequence Synthetic peptide
ILPRGG

SEQ ID NO: 44 - protein, 6 aa, Artificial Sequence
  Description of Artificial Sequence Synthetic peptide
LPRGGS

SEQ ID NO: 45 - protein, 6 aa, Artificial Sequence
  Description of Artificial Sequence Synthetic peptide
PRGGSV

SEQ ID NO: 46 - protein, 6 aa, Artificial Sequence
  Description of Artificial Sequence Synthetic peptide
RGGSVL

SEQ ID NO: 47 - protein, 8 aa, Artificial Sequence
  Description of Artificial Sequence Synthetic peptide
XPKSVILC

SEQ ID NO: 48 - protein, 8 aa, Artificial Sequence
  Description of Artificial Sequence Synthetic peptide
XSKVILPC

SEQ ID NO: 49 - protein, 8 aa, Artificial Sequence
  Description of Artificial Sequence Synthetic peptide
XKVILPRC

SEQ ID NO: 50 - protein, 8 aa, Artificial Sequence
  Description of Artificial Sequence Synthetic peptide
XVILPRGC

SEQ ID NO: 51 - protein, 8 aa, Artificial Sequence
  Description of Artificial Sequence Synthetic peptide
XILPRGGC

SEQ ID NO: 52 - protein, 8 aa, Artificial Sequence
  Description of Artificial Sequence Synthetic peptide
XLPRGGSC

SEQ ID NO: 53 - protein, 8 aa, Artificial Sequence
  Description of Artificial Sequence Synthetic peptide
XPRGGSVC

SEQ ID NO: 54 - protein, 8 aa, Artificial Sequence
  Description of Artificial Sequence Synthetic peptide
XRGGSVLC

SEQ ID NO: 55 - protein, 6 aa, Artificial Sequence
  Description of Artificial Sequence Synthetic peptide
KRGGSV

SEQ ID NO: 56 - protein, 6 aa, Artificial Sequence
  Description of Artificial Sequence Synthetic peptide
PKGGSV

SEQ ID NO: 57 - protein, 6 aa, Artificial Sequence
  Description of Artificial Sequence Synthetic peptide
PRKGSV

SEQ ID NO: 58 - protein, 6 aa, Artificial Sequence
  Description of Artificial Sequence Synthetic peptide
PRGKSV

SEQ ID NO: 59 - protein, 6 aa, Artificial Sequence
  Description of Artificial Sequence Synthetic peptide
PRGGKV

SEQ ID NO: 60 - protein, 6 aa, Artificial Sequence
  Description of Artificial Sequence Synthetic peptide
PRGGSK

SEQ ID NO: 61 - protein, 6 aa, Artificial Sequence
  Description of Artificial Sequence Synthetic peptide
PRGXSK

SEQ ID NO: 62 - protein, 6 aa, Artificial Sequence
  Description of Artificial Sequence Synthetic peptide
VILPRG

SEQ ID NO: 63 - protein, 6 aa, Artificial Sequence
  Description of Artificial Sequence Synthetic peptide
PRGGSV

SEQ ID NO: 64 - protein, 6 aa, Artificial Sequence
  Description of Artificial Sequence Synthetic peptide
KRGGSV

SEQ ID NO: 65 - protein, 6 aa, Artificial Sequence
  Description of Artificial Sequence Synthetic peptide
PKGGSV

SEQ ID NO: 66 - protein, 6 aa, Artificial Sequence
  Description of Artificial Sequence Synthetic peptide
PRKGSV

SEQ ID NO: 67 - protein, 6 aa, Artificial Sequence
  Description of Artificial Sequence Synthetic peptide
PRGKSV

SEQ ID NO: 68 - protein, 6 aa, Artificial Sequence
  Description of Artificial Sequence Synthetic peptide
PRGGKV

SEQ ID NO: 69 - protein, 6 aa, Artificial Sequence
  Description of Artificial Sequence Synthetic peptide
PRGGSK

SEQ ID NO: 70 - protein, 6 aa, Artificial Sequence
  Description of Artificial Sequence Synthetic peptide
PRXGSK

SEQ ID NO: 71 - protein, 10 aa, Artificial Sequence
  Description of Artificial Sequence Synthetic peptide
PRGXSKXXXX

SEQ ID NO: 72 - protein, 10 aa, Artificial Sequence
  Description of Artificial Sequence Synthetic peptide
LPRGGSVLVT

SEQ ID NO: 73 - protein, 12 aa, Artificial Sequence
  Description of Artificial Sequence Synthetic peptide
XPSKVILPRGGC

SEQ ID NO: 74 - protein, 6 aa, Artificial Sequence
  Description of Artificial Sequence Synthetic peptide
PRGNSK

SEQ ID NO: 75 - protein, 6 aa, Artificial Sequence
  Description of Artificial Sequence Synthetic peptide
PRGFSK

SEQ ID NO: 76 - protein, 6 aa, Artificial Sequence
  Description of Artificial Sequence Synthetic peptide
PRGVSK

SEQ ID NO: 77 - protein, 6 aa, Artificial Sequence
  Description of Artificial Sequence Synthetic peptide
PRGDSK

SEQ ID NO: 78 - protein, 6 aa, Artificial Sequence
  Description of Artificial Sequence Synthetic peptide
PRGRSK

SEQ ID NO: 79 - protein, 5 aa, Artificial Sequence
  Description of Artificial Sequence Synthetic peptide
TDGEA

SEQ ID NO: 80 - protein, 12 aa, Artificial Sequence
  Description of Artificial Sequence Synthetic peptide
XPRGGSVLVTGC

SEQ ID NO: 81 - protein, 12 aa, Artificial Sequence
  Description of Artificial Sequence Synthetic peptide
XQTSVSPSKVIC

SEQ ID NO: 82 - protein, 12 aa, Artificial Sequence
  Description of Artificial Sequence Synthetic peptide
XITDGEATDSGC

SEQ ID NO: 83 - protein, 12 aa, Artificial Sequence
  Description of Artificial Sequence Synthetic peptide
XDLSTSLDDLRC

(19) United States
(12) Patent Application Publication
(10) Pub. No.: US 2004/0037775 A1
(43) Pub. Date: Feb. 26, 2004

(54) Leukocyte internalized peptide-drug conjugates

(76) Inventors: Teruna J. Siahaan, Lawrence, KS (US); Helena Yusuf-Makagiansar, San Diego, CA (US); Meagan Anderson, Kansas City, MO (US); Rong?quot;Christine?quot; Xu, Tustin, CA (US)

Correspondence Address: Narinder S. Banait, Esq., Frenwick & West LLP, Silicon Valley Center, 801 California Street, Mountain View, CA, 94041, US

(21) Appl. No.: 10/464,302

(22) Filed: Jun. 17, 2003

Related U.S. Application Data
(63) Continuation-in-part of application No. 09/629,719, filed on Aug. 1, 2000.

(51) Int. Cl. A61K 49/00; A61K 38/12; A61K 38/10; C07K 7/08

(52) U.S. Cl. 424/9.1; 514/9; 530/317; 530/324

(57) ABSTRACT

The invention discloses compositions and methods useful for treating and preventing autoimmune diseases. The compositions and methods utilize peptides that are cell-specific. The peptides are conjugated to drugs. The peptide-drug conjugate can be internalized by the targeted cells thereby allowing for cell-specific delivery of the drug.

### CROSS-REFERENCE TO RELATED APPLICATION

This application is a continuation-in-part of patent applications Ser. No. 09/629,719 filed Aug. 1, 2000, from which priority is claimed and which application is incorporated herein by reference in its entirety.

### FIELD OF THE INVENTION

The present invention relates to peptides, particularly peptides derived from intracellular adhesion molecule-1 and lymphocyte function-associated antigen-1, and the conjugation of the peptides with drugs for cell-specific drug delivery.

### BACKGROUND OF THE INVENTION

Leukocyte-related diseases often result from aberrant immune responses including reactions of leukocytes to “self” antigens. Such reactions contribute to autoimmune diseases including rheumatoid arthritis, insulin-dependent diabetes, mellitus, lupus erythematosis, and multiple sclerosis. Similarly, organ transplantation rejection results from leukocyte attack, specifically from T-cells. Accordingly inhibition of T-cell actions and their subsequent destruction aids in combating such diseases.

One way to modulate leukocyte immune response utilized inhibitors of ICAM-1/LFA-1 receptor interaction. For example, monoclonal antibodies (mAbs) to ICAM-1 and LFA-1 have been utilized to generate tolerance in immune response disorders such as allograft rejection (Kato et al. (1996) Ann. Surg. 223: 94-100; Nakamura et al. (1996) Transplantation 62: 547-552), rheumatoid arthritis (Davis et al. (1995) J. Immunol. 154: 3525-3537), and autoimmune encephalomyelitis (Willenborg et al. (1996) J. Immunol. 157: 1973-1980). Despite the encouraging clinical results in inducing tolerances, such mAbs may be potentially immunogenic and trigger an effectiveness-limiting immunity. In addition, the formulation of antibodies is challenging and costly. Another way to modulate immune response utilizes small peptide fragments derived from ICAM-1 and LFA-1 sequences which inhibit ICAM-1/LFA-1 interaction (Ross et al. (1992) J. Biol. Chem 267: 8537-8543; Fecondo et al. (1993) AIDS Res. Hum. Retrovirus 9: 733-740; Benedict et al. (1994) U.S. Pat. Nos. 5, 843,885 and 5,863,889; and Siahaan et al. (1996) in Peptides: Chemistry, Structure and Biology (Kaumaya PTP and Hodges RS eds) pp 792-793, Mayflower Scientific, Endgland). These peptides may have better physicochemical stability than antibodies and may not possess any immunogenic properties. It has also been shown that a cyclic peptide (cIBR) derived from the sequence of ICAM-1 inhibits ICAM-1/LFA-1 interactions (Siahaan et al. (1996)).

Furthermore, despite the ability to inhibit ICAM-1/LFA-1 interactions and attendant leukocyte-related diseases through treatment with antibodies, such treatments are typically ineffective over the long term due to their transient nature. Additionally, once the mAbs invoke an immune response, their effectiveness is severely limited.

When toxic drugs are used to kill the leukocytes and combat leukocyte-related diseases, many adverse side effects are encountered. These side effects include the non-selective killing of cells in addition to targeted cells as well as the suppression of the proliferation of healthy cells. Therefore, new methods which selectively target drugs to cells involved in the disease process will be beneficial to patients. For example, selectively targeting cytotoxic drugs to leukocytes will reduce drug toxicity and increase drug efficacy.

### SUMMARY OF THE INVENTION

The present invention provides methods and compositions of peptides conjugated to moieties, such as drugs and methods of using the peptide-drug conjugates. The peptide-drug conjugates of the invention can be used for treating and preventing immune diseases, such as autoimmune diseases. These peptide-drug conjugates can be delivered alone or in combination with additional agents.

Accordingly, in one aspect, the subject invention is directed to compounds of formula P-L-M where P is a peptide comprising about 4 to 12 contiguous amino acid residues from an ICAM-1 or LFA-1 protein sequence, L is a direct bond or a linker having from 1 to about 20 carbon atoms, and M is a reporter molecule, a dye, or a drug. The peptide can be a linear peptide, and further comprise Xaa and Cys as terminal amino acids, wherein Xaa is Pen or Cys that can be used to cyclize the peptide. The peptide can derived from LFA-1, such as the insert (I) domain, the cation binding domain V and VI, or the I-domain like region of LFA-1. Alternatively, the peptide can be derived from ICAM-1, such as the D1 region of ICAM-1. The linker L can be a direct bond, or can be 4 amino acid residues. The moiety M can be a drug selected from the group consisiting of methotrexate, lovastatin, taxol, ajmalicine, vinblastine, vincristine, cyclophosphamide, fluorouracil, idarubicin, ifosfamide, irinotecan, 6-mercaptopurine, metomycins, mitoxantrone, paclitaxel, pentostatin, plicamycin, topotecan, fludarabine, etoposide, doxorubicin, doxetaxel, danorubicin, albuterol, and propidium. Preferably, the drug is methotrexate, fluorouracil or paclitaxel.

In another aspect, the invention provides compounds of formula cPRGX_{bb}SK or cPRX_{bb}GSK, where X_{bb} is a neutral, hydrophobic or charged residue selected from the group consisting of N(Asn), F(Phe), V(Val), D(Asp), or R(Arg).

In another aspect, the invention provides compounds of formula:

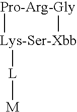

wherein X_{bb} is a neutral, hydrophobic or charged residue selected from the group consisting of Asn, Phe, Val, Asp, or Arg; L is a direct bond or a linker having from about 1 to about 20 carbon atoms; and M is a reporter molecule, a dye, or a drug. Preferably, X_{bb} is Asn or Asp, L is a direct bond or a linker comprising 4 amino acid residues. The drug can be methotrexate or Taxol.

In another aspect, the invention provides methods of treating a subject, the method comprising administering a therapeutically effective amount of a compound of formula P-L-M wherein P is a peptide comprising about 4 to 12 contiguous amino acid residues from an ICAM-1 or LFA-1 protein sequence, L is a direct bond or a linker having from 1 to 20 carbon atoms, and M is a reporter molecule, a dye, or a drug in admixture with at least one pharmaceutically acceptable carrier. The drug can be selected from the group consisiting of methotrexate, lovastatin, taxol, ajmalicine, vinblastine, vincristine, cyclophosphamide, fluorouracil, idarubicin, ifosfamide, irinotecan, 6-mercaptopurine, metomycins, mitoxantrone, paclitaxel, pentostatin, plicamycin, topotecan, fludarabine, etoposide, doxorubicin, doxetaxel, danorubicin, albuterol, and propidium. The subject can be a mammal, such as human, mouse, rat, horse, and the like.

The invention thus provides methods for treating or preventing immune diseases, such as autoimmune diseases in a mammalian subject in need thereof, the method comprising administering a pharmaceutically effective amount of a peptide-drug conjugate or salts, or solvates thereof, to the subject. The disease can be arthritis, such as rheumatoid arthritis, or psoriac arthritis, multiple sclerosis, type-I diabetes, psoriasis, lupus erythematosis, cancer, asthma, Crohn's disease, ulcerative colitis, pemphigus vulgaris, pemphigoid, myasthenia gravis, HIV-infections, allergies, and epidermolysis. Further, the invention provides methods for administering an additional active agent. The peptide-drug conjugates of the invention are administered in a pharmaceutical composition containing a pharmaceutically acceptable excipient. The excipient is suitable for oral administration. Thus, the composition is in the form of a tablet, a capsule, or a soft-gel capsule. In addition, the excipient is liquid suited to intravenous, intramuscular, or subcutaneous administration. Further, the excipient is suited to transdermal administration, or buccal administration.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF THE DRAWINGS

The patent or application file contains at least one drawing executed in color. Copies of this patent with color drawing(s) will be provided by the Patent and Trademark Office upon request and payment of the necessary fee.

FIG. 1 illustrates a model of methotrexate-cyclo-Leu Pro Arg Gly Gly Ser Val Leu Val Thr (MTX-cIBR) binding to DHFR using the predetermined position of MTX complexed to DHFR.

FIG. 2: 2A illustrates the simulated binding of the linear 10 amino acid residues (Ile²³⁷-Gly²⁴⁶) of the LFA-1 derived peptide cLAB.1 to the D1-domain of ICAM-1. 2B illustrates the simulated binding of cyclic-ITDGEA ) to the D1-domain of ICAM-1.

FIG. 3 illustrates Western blot analysis of ICAM-1 in Calu-3 cell lysates. M=Molecular weight markers; 1P=pellet from 12,000 g centrifugation; 2P=pellet from 65,000 g centrifugation; S=supernatant from 65,000 g centrifugation; rI=soluble recombinant hICAM-1 standard.

FIG. 4 illustrates the effects of peptides blocking on IFN-γ-induced Calu-3 cell-monolayers to the adherence of PMA-activated Molt-3 T-cells. The cyclic I-domain peptide (cLAB.L) significantly reduces the adherence of T-cells to epithelial monolayers while no significant effect is given by the domain V peptide (cLAB.2L). *P<0.05 and **P<0.01 as compared with control.

FIG. 5 illustrates the effect of LFA-1 derived peptide, MTX and MTX-peptide on the growth and cytotoxicity of HCAEC (A) and Molt-3 T-cells (B). Bar 1 to 6 of each compound represent the concentration of 0.1, 1, 10, 50, 100, and 500 μM, respectively. Based on the relative amount of the remaining cellular polynucleic acids (PNA), the qualitative effect of the compound, presented as relative cytotoxicity, falls within the grades of causing partial growth inhibition (a), total growth inhibition (b) or net cell killing (c).

FIG. 6: 6A illustrates the Thymidine synthase (TS) inhibition during continuous exposure assay. Slopes represent the rate of ³H₂O produced in 1 h by TS after 4 h incubation with either MTX or MTX-peptides where the peptides are derived from ICAM-1, and in untreated control cells. 6B illustrates the comparison of wash-out (4 h+4 h DFM) and continuous exposure (4 h) of the ability of MTX and MTX-peptide conjugates to inhibit TS.

FIG. 7 illustrates the results of ELISA assay to quantify TNF-α production by activated and resting human PBL treated with MTX or MTX-peptide conjugates, and untreated control cells.

FIG. 8 illustrates the effect of peptide, MTX, and MTX-peptide on IL-6 (A) and IL-8 (B) production in HCAEC. The cell monolayers were cultured in vitro with the test compounds (0.001 to 100 μM) in the presence of TNF-α for 24 h. Results are expressed as the percentage of cytokine relative to the positive control or non-treated monolayers (the baseline 100% cytokine production. The control levels (mean±SE) were as follows: IL-6: 3.4±0.17 ng/mL, IL-8: 199.5±9.13 ng/mL.

### DETAILED DESCRIPTION

I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology, within the skill of the art.

The term “modulator” means a molecule that interacts with a target. The interactions include, but are not limited to, agonist, antagonist, and the like, as defined herein.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proiine: Pro (P) | Serine: Ser (S) |
| Threonine: Thr (T) | Tryptophan: Trp (W) |
| Tyrosine: Tyr (Y) | Valine: Val (V) |

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations arising with hosts that produce the proteins or errors due to PCR amplification.

As used herein, an “analogue” or “derivative” is a compound, e.g., a peptide, having more than about 70% sequence but less than 100% sequence similarity with a given compound, e.g., a peptide. Such analogues or derivatives may be comprised of non-naturally occurring amino acid residues, including by way of example and not limitation, homoarginine, ornithine, penicillamine, and norvaline, as well as naturally occurring amino acid residues. Such analogues or derivatives may also be composed of one or a plurality of D-amino acid residues, and may contain non-peptide interlinkages between two or more amino acid residues.

As used herein, the terms “label”, “detectable label”, and “reporter molecule” refer to a molecule capable of being detected, including, but not limited to, radioactive isotopes, fluorescers, chemiluminescers, chromophores, magnetic resonance agents, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

The term “alkyl” means the monovalent branched or unbranched saturated hydrocarbon radical, consisting solely of carbon and hydrogen atoms, having from one to twelve carbon atoms inclusive, unless otherwise indicated. Examples of alkyl radicals include, but are not limited to, methyl, ethyl, propyl, isopropyl, butyl, isobutyl, sec-butyl, tert-butyl, pentyl, n-hexyl, octyl, dodecyl, and the like.

The term “alkylene” as used herein means the divalent linear or branched saturated hydrocarbon radical, consisting solely of carbon and hydrogen atoms, having from one to eight carbon atoms inclusive, unless otherwise indicated. Examples of alkylene radicals include, but are not limited to, methylene, ethylene, trimethylene, propylene, tetramethylene, pentamethylene, ethylethylene, and the like.

The term “alkenylene” means the divalent linear or branched unsaturated hydrocarbon radical, containing at least one double bond and having from two to eight carbon atoms inclusive, unless otherwise indicated. The alkenylene radical includes the cis or trans ((E) or (Z)) isomeric groups or mixtures thereof generated by the asymmetric carbons. Examples of alkenylene radicals include, but are not limited to ethenylene, 2-propenylene, 1-propenylene, 2-butenyl, 2-pentenylene, and the like.

The term “aryl” means the monovalent monocyclic aromatic hydrocarbon radical consisting of one or more fused rings in which at least one ring is aromatic in nature, which can optionally be substituted with hydroxy, cyano, lower alkyl, lower alkoxy, thioalkyl, halogen, haloalkyl, hydroxyalkyl, nitro, alkoxycarbonyl, amino, alkylamino, dialkylamino, aminocarbonyl, carbonylamino, aminosulfonyl, sulfonylamino, and/or trifluoromethyl, unless otherwise indicated. Examples of aryl radicals include, but are not limited to, phenyl, naphthyl, biphenyl, indanyl, anthraquinolyl, and the like.

The term “halogen” as used herein refers to fluoro, bromo, chloro and/or iodo.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a peptide-drug conjugate disclosed herein required to provide a clinically significant decrease in the symptoms of an autoimmune disease, such as those resulting from rheumatoid arthritis. An appropriate “effective” amount in any individual case may be determined by one of ordinary skill in the art using routine experimentation.

As used herein, the terms “treat” or “treatment” are used interchangeably and are meant to indicate a postponement of development of an autoimmune disease and/or a reduction in the severity of such symptoms that will or are expected to develop. The terms further include ameliorating existing symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The term “pharmaceutically acceptable salt” of a compound means a salt that is pharmaceutically acceptable and that possesses the desired pharmacological activity of the parent compound. Such salts, for example, include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic. acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;

(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

II. Overview

The present invention provides methods and compositions for the treatment of immunological disorders, including but not limited to, autoimmune diseases, such as rheumatoid arthritis, multiple sclerosis (“MS”), psoriasis, cancers, and viral infections such as HIV, HCV, and other viral infections. In one aspect of the invention, cell-specific peptides are identified and described. The cell-specific peptides can be from about 3 to about 30 amino acids in length, and can be derived from the ICAM-1 and LFA-1 sequences. The peptides can thus be specific for leukocytes and can be used for treating leukocyte-related diseases. The selected peptides can be either linear or cyclic, and can be substituted with non-natural occurring amino-acids. The peptides can be conjugated to a moiety. The conjugation can be either through a direct bond or via linkers having between 1 and 20 carbon atoms. The moiety can be a label, a drug, an intercalator, or another peptide or protein, such as an antibody.

In one aspect of the invention, linear or cyclized peptides having about 4 amino acids to about 12 amino acids are conjugated to drugs, such as cytotoxic drugs. The peptide-drug conjugates are internalized by the targeted cells. The conjugated drug can act on the targeted biological mechanism. The peptides thus provide a means of cell-specific drug delivery system, and the conjugate can be used as therapeutic agents for the treatment of diseases.

In another aspect, the invention provides compositions of compounds of formula P-L-M, wherein P is a peptide having about 4 amino acid to about 12 amino acid residues, L is either a direct bond or a linker having about 1 to about 20 carbon atoms, and M is a moiety, such as a reporter group, including fluorescent compounds, an intercalator or a drug.

III. Peptide Selection

In accordance with the present invention, peptides of about 3 to about 30 amino acids in length useful for preventing and treating disease conditions are described. The peptides can be used in methods and compositions for cell specific treatment of diseases.

The peptides for use in the invention are selected from the sequence of ICAM-1 (accession no. AAE 18917) or LFA-1 (accession nos. AAE 18915 or AAE 18916). The amino acid residue sequences of the parent integrin LFA-1 includes the β- or CD18 subunit (accession no. 18915) and the α- or CD11a subunit (accession no. AAE 18916). The peptides selected from the parent proteins can be linear or cyclic and can be from about 3 to about 30 amino acid residues in length, preferably about 4 to about 15 amino acid residues in length, and more preferably about 4 to about 12 amino acid residues in length, or any integer in between. Thus, the peptides can be 4, 5, 6, 7, 8, 9, 10, 11 or 12 amino acid residues in length.

LFA-1 Peptides

In one aspect of the invention, the peptides of about 4 to 12 contiguous amino acid residues are selected from the sequence of the LFA-1 protein. The peptides are selected such that they tile across the entire sequence of the parent LFA-1 protein with successive overlapping sequences of 0, 1, 2, 3, 4, 5, or 10 amino acid residues, or any other integral amino acid interval. Thus, for example, using LFA-1 sequence of accession number AAE 18916, the first peptide can have the sequence corresponding to the contiguous position 1-10, the second can be from position 8-17, the third can be from position 15-24, and so on such that all peptides are 10 amino acid residues in length with an overlap of 3 amino acid residues. The peptides thus selected can be used as a library. As will be evident to one of skill in the art, the library can contains peptides of different lengths and different overlap.

In another aspect, the peptides for use in the present invention are selected from particular regions of the LFA-1 sequences, such as the functional domains, signal sequences or sequence repeat regions. For example, LFA-1 has at least three binding regions: insert (I) domain that is located in the N-terminal region of the α-subunit of LFA-1, and is composed of approximately 200 amino acid residues; the cation binding domain V and VI; and the I-domain-like region of the β₂ subunit. Thus, peptides of about 4 to about 30 amino acid residues can be selected from the binding region of LFA-1. In one aspect, the peptides are selected from the binding region of the LFA-1 protein. Thus, for example, the peptide LAB, having the sequence ITDGE ATDSG NIDAA KDIIY IIGI (SEQ ID No. 1), derived from the I-domain of the α-subunit of LFA-1, and corresponding to the contiguous sequences Ile²³⁷-Ile²⁶¹ can be selected. In another example, the peptide LAB.2, having the sequence Gly Val Asp Val Asp Gln Asp Gly Glu Thr Glu Leu Ile Gly Ala Pro Leu Phe Tyr Gly GIu Gln Arg Gly (SEQ ID No. 2), corresponding to sequences Gly⁴⁴¹-Gly⁴⁶⁴ can be derived from domain V of the α-subunit of LFA-1. In yet another example, the peptide LBE, corresponding to sequence Asp Leu Ser Tyr Ser Leu Asp Asp Leu Arg Asn Val Lys Lys Leu Gly Gly Asp Leu Leu Arg Ala Leu Asn Glu (SEQ ID No. 3) can be derived from the I-domain like region of the β-subunit of LFA. The peptides LAB and LAB.2 have previously been shown potent activity in inhibiting homotypic T-cell adhesion by 30-52%.

In one aspect of the invention, the peptides LAB, LAB.2, and LBE are selected and covalently linked, optionally using linker, to form an ICAM-1 binding peptide. In another aspect, peptides of about 4 to about 12 consecutive amino acid residues are selected from each of LAB, LAB.2, and LBE. The selected peptides are then covalently linked, optionally using linker, to form an ICAM-1 binding peptides. As explained earlier, the peptides can also be selected such that they tile across the binding region of the LFA-1 protein, with successive overlapping sequences of 0, 1, 2, 3, 5, or 10 amino acid residues, or any other integer residue interval.

In yet another aspect of the invention, peptides of about 4 amino acid residues to about 12 amino acid residues that tile across LAB and LAB.2 are selected. Thus, for example, the peptides derived from LAB can be LAB.L (ITDGE ATDSG) (SEQ ID No. 4); ITDGEA (SEQ ID No. 5); TDGEAT (SEQ ID No. 6); DGEATD (SEQ ID No. 7); GEATDS (SEQ ID No. 8); EATDSG (SEQ ID No. 9); and DGEA (SEQ ID No. 10), and the like. Similarly, the peptides derived from LAB.2 can be LAB.2L (Gly Val Asp Val Asp Glp Asp Gly Glu Thr) (SEQ ID No. 11); LAB.2C (Gly Glu Thr Glu Leu Ile Gly Ala Pro Leu) (SEQ ID No. 12); and LAB.2R (Ala Pro Leu Tyr Gly Glu Gln Arg Gly Lys) (SEQ ID No. 13).

In another aspect, peptides of about 4 amino acid residues to about 12 amino acid residues that tile across LFA-1, LAB and LAB.2 are selected, and further modified. For example, any of the amino acid residues, the N-terminus and/or the C-terminus can be modified. The modification can be such that the peptides have longer half-lives in a subject, have altered physical properties, such as the ability to form β-sheets or possess particular functional groups that can be chemically modified, and the like. Thus, for example, the peptides can be cyclized. In one aspect, amino acid residues are added to the N-terminus and the C-terminus, where the peptide thus modified is capable of forming a cyclized peptide. Thus, the peptides derived from LAB can be LAB.L (Xaa-ITDGE ATDSG-Cys) (SEQ ID No. 14); Xaa-ITDGEA-Cys (SEQ ID No. 15); Xaa-TDGEAT-Cys (SEQ ID No. 16); Xaa-DGEATD-Cys (SEQ ID No. 17); Xaa-GEATDS-Cys (SEQ ID No. 18); Xaa-EATDSG-Cys (SEQ ID No. 19); and Xaa-DGEA-Cys (SEQ ID No. 20), and the like, wherein Xaa is Cys or Pen, and are optionally added to form cyclic peptides. Similarly, the peptides derived from LAB.2 can be LAB.2L (Xaa-Gly Val Asp Val Asp Glp Asp Gly Glu Thr-Cys) (SEQ ID No. 21); LAB.2C (Xaa-Gly Glu Thr Glu Leu Ile Gly Ala Pro Leu-Cys) (SEQ ID No. 22); and LAB.2R (Xaa-Ala Pro Leu Tyr Gly Glu Gln Arg Gly Lys-Cys) (SEQ ID No. 23).

In another aspect of the invention, the peptides are selected such that each contains at least one of Asp, Glu, Thr, or Ser amino acid residues, preferably the Asp amino acid residues. Thus, for example, Asp²³⁹ Glu²⁴¹ Thr²⁴³ and Ser²⁴⁵ and 1-10 contiguous amino acid residues on either side of the residue can be selected. The peptide sequences thus selected can be, for example, SEQ ID Nos. 1, 4, 5, 6, 7, or 10. In another aspect, the peptides are selected such that the sequences include the amino acid residues IT, such as Ile²³⁷ Thr²³⁸; amino acid residues TD, such as Thr²⁴³ Asp²⁴⁴; amino acid residues ITD, such as Ile²³⁷Thr²³⁸ Asp²³⁹ (SEQ ID No. 24); or amino acid residues ITDG, such as Ile²³⁷Thr²³⁸ Asp²³⁹ Gly²⁴⁰ (SEQ ID No. 25). Preferably, the peptide sequences contain the amino acid residues IT or ITD.

ICAM-1 Peptides

In one aspect of the invention, the peptides are selected such that the contiguous amino acid sequences tile across the entire ICAM-1 sequence of the parent proteins with successive overlapping sequences of 0, 1, 2, 3, 4, 5, or 10 amino acid residues, or any other integral amino acid interval. Thus, for example, using ICAM-1 sequence of accession number AAE 18917, the first peptide can have the sequence corresponding to the contiguous position 1-10, the second can be from position 8-17, the third can be from position 15-24, and so on. The peptides thus selected can be used as a library, where the library can contains peptides of different lengths.

In another aspect, the peptides for use in the present invention are selected from particular regions of the ICAM-1 sequences, such as the functional domains, signal sequences or sequence repeat regions. For the ICAM-1 protein, the D1 region is thought to be the binding region. In this aspect of the invention, the peptides are selected from the D1 region of the ICAM-1 protein. The peptides can be from about 3 amino acid residues to about 30 amino acid residues in length, preferably from about 4 amino acid residues to about 12 amino acid residues in length, and can be selected such that the peptides tile across the D-1 region of the ICAM-1 protein, with successive overlapping sequences of 0, 1, 2, 3, 5, or 10 amino acid residues, or any other integer residue interval. Thus, for example, the peptide IB, having the sequence Gln Thr Ser Val Ser Pro Ser Lys Bal Ile Leu Pro Arg Gly Gly Ser Val Leu Val Thr Gly (SEQ ID No. 26), or the peptide IE, having the sequence Asp Gly Pro Lys Leu Leu Gly Ile Glu Thr Pro Leu Pro Lys Lys Glu Leu Leu Pro Gly Asn Asn Arg Lys (SEQ ID No. 27) can be selected.

In yet another aspect of the invention, peptides of about 4 amino acid residues to about 12 amino acid residues that tile across IB and IE are selected. Thus, for example, the peptides derived from IB can be Pro Ser Lys Val Ile Leu Pro Arg Gly Gly (IBC; SEQ ID No. 28), Gln Thr Ser Val Ser Pro Ser Lys Val Ile (IBL; SEQ ID No. 29), Leu Pro Arg Gly Gly Ser Val Leu Val Thr (IBR; SEQ ID No. 30). Further, the peptides derived from IE can be Glu Thr Pro Leu Pro Lys Lys Glu Leu Leu (IEC; SEQ ID No. 31), Asp Gln Pro Lys Leu Leu Gly Ile Glu Thr (IEL; SEQ ID No. 32), Glu Leu Leu Leu Pro Gly Asn Asn Arg Lys (IER; SEQ ID No. 33), and the like.

As described above, the peptides can be modified. In one aspect, amino acid residues are added to the N-terminus and the C-terminus, where the peptide thus modified is capable of forming a cyclized peptide. Thus, the peptides derived from ICAM-1 can be the modified IBC peptide Xaa Pro Ser Lys Val Ile Leu Pro Arg Gly Gly Cys (SEQ ID No. 33), the modified IBL peptide Xaa Gln Thr Ser Val Ser Pro Ser Lys Val Ile Cys (SEQ ID No. 34), the modified IBR peptide Xaa Leu Pro Arg Gly Gly Ser Val Leu Val Thr Cys (SEQ ID No. 35), the modified IEC peptide Xaa Glu Thr Pro Leu Pro Lys Lys Glu Leu Leu Cys (SEQ ID No. 36), the modified IEL peptide Xaa Asp Gln Pro Lys Leu Leu Gly Ile Glu Thr Cys (SEQ ID No. 37), the modified IER peptide Xaa Glu Leu Leu Leu Pro Gly Asn Asn Arg Lys Cys(SEQ ID No. 38), and the like wherein Xaa can be Cys or Pen.

Alternatively, the peptides can be about 6 amino acid residues in length, and selected to tile across IB or IE with an overlap of 1, 2, 3, 4, or 5 amino acid residues. Thus, the peptides PKSVIL (SEQ ID No. 39), SKVILP (SEQ ID No. 40), KVILPR (SEQ ID No. 41), VILPRG (SEQ ID No. 42), ILPRGG (SEQ ID No. 43), LPRGGS (SEQ ID No. 44), PRGGSV (SEQ ID No. 45), and RGGSVL (SEQ ID No. 46) can be selected from the sequence of IB (SEQ ID No.26). As described above, the peptides can be further modified by optionally adding an amino acid residue to each termini of the peptide. Thus, the peptides Xaa-PKSVIL-Cys (SEQ ID No. 47), Xaa-SKVILP-Cys (SEQ ID No. 48), Xaa-KVILPR-Cys (SEQ ID No. 49), Xaa-VILPRG-Cys (SEQ ID No. 50), Xaa-ILPRGG-Cys (SEQ ID No. 51), Xaa-LPRGGS-Cys (SEQ ID No. 52), Xaa-PRGGSV-Cys (SEQ ID No. 53), and Xaa-RGGSVL-Cys (SEQ ID No. 54) can be selected from the sequence of IB (SEQ ID No.26). As one of skill in the art will recognize, the length of the peptides and the overlap can vary.

Peptide Analogues

It is well know to those skilled in the art that modifications can be made to the peptides of the invention to provide them with altered properties. As used herein the term “amino acid” refers to either natural and/or unnatural or synthetic amino acids, including glycine and both the D- or L-optical isomers, and amino acid analogs and peptidomimetics. Thus, the peptides of the invention can be all D-isomer, all L-isomer, or a combination thereof where the peptides contain at least one D- or at least one L-amino acid residue. Peptides of the invention can be modified to include unnatural amino acids. Thus, the peptides may comprise D-amino acids, a combination of D- and L-amino acids, and various “designer” amino acids (e.g., β-methyl amino acids, Cα-methyl amino acids, and Nα-methyl amino acids, and the like) to convey special properties to peptides. Additionally, by assigning specific amino acids at specific coupling steps, peptides with α-helices, β-turns, β-sheets, γ-turns, and cyclic peptides can be generated.

In one aspect of the invention, the peptide selected contains at least one D-amino acid. Any of the amino acid residues can be changed to the D-isomer. Thus, for example, if the peptide selected is PRGGSV (SEQ ID NO. 45), then at least one of the amino acid residues, i.e. P, R, G, S, or V can be D-isomer, or two of the amino acid residues can be the D-isomer, or 3 or more of the amino acid residues can be the D-isomer. It is preferable that one of the terminal amino acid residues, preferably the C-terminus, be modified to have the D-isomer amino acid residue.

In an aspect of the invention, subunits of peptides that confer useful chemical and structural properties can be selected. For example, peptides comprising D-amino acids will be resistant to L-amino acid-specific proteases in vivo. The peptides, selected according to the criteria discussed in detail above, can be modified with D-amino acids and can be synthesized with the amino acids aligned in reverse order to produce the peptides of the invention as retro-inverso peptides. Thus, for example, SEQ ID No. 15 can be modified such that the amino acid residue T has the D-conformation, or the amino acid residues I and T have the D-conformation, or all the amino acids are the D-isomer. In addition, the present invention envisions preparing peptides that have well-defined structural properties, and the use of peptidomimetics, and peptidomimetic bonds, such as ester bonds, to prepare peptides with novel properties. In another aspect, a peptide may be generated that incorporates a reduced peptide bond, i.e., R₁—CH₂ NH—R₂, where R₁, and R₂ are amino acid residues or alkyl, aryl, or heteroalkyl substituents. A reduced peptide bond can be introduced as a dipeptide subunit, thereby making the peptide resistant to peptide bond hydrolysis, such as, protease activity, thereby extending the in vivo half-live due to resistance to metabolic breakdown, or protease activity.

In another aspect, non-classical amino acids can be incorporated in the peptides of the invention in order to introduce particular conformational motifs. Non-classical amino acids include 1,2,3,4-tetrahydroisoquinoline-3-carboxylate; (2S,3S)-methyl-phenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; histidine isoquinoline carboxylic acid; and HIC (histidine cyclic urea). In yet another aspect, amino acid analogs and peptidomimetics can be incorporated into the peptides of the invention to induce or favor specific secondary structures. Such analogs and peptidomimetics include LL-Acp (LL-3-amino-2-propenidone-6-carboxylic acid), and conformationally restricted mimetics of beta turns and beta bulges, described in U.S. Pat. No. 5,440,013 to Kahn.

In this aspect of the invention, the sequence PRGGSV (SEQ ID NO. 45) can be modified such that at least one of the amino acid residues is replaced by the lysine (K) residue. Thus, the sequence can be KRGGSV (SEQ ID NO. 55), PKGGSV (SEQ ID NO. 56), PRKGSV (SEQ ID NO. 57), PRGKSV (SEQ ID NO. 58), PRGGKV (SEQ ID NO. 59), or PRGGSK (SEQ ID NO. 60). The peptides of SEQ ID Nos. 55-60 can be cyclized by forming an amide bond between the first residue and the last residue to give cyclized peptides. The incorporation of the lysine amino acid residue conveniently provides chemically reactive groups or a handle to which can be attached a linker and a moiety, such as a drug. Thus, in this aspect of the invention, any amino acid residue that can provide a chemically reactive group capable of further elaboration can be used.

In yet another aspect of the invention, the selected sequence is modified so that at least one of the amino acid residues is replaced by a hydrophilic amino acid residue. The hydrophilic amino acid residue can be acidic, basic, or polar. The acidic amino acid residue has a negative charge due to loss of a H⁺ ion at physiological pH and the residue is attracted by aqueous solution so as to seek the surface positions in the conformation of a peptide in which it is contained when the peptide is in aqueous medium at physiological pH. Naturally occurring acidic amino acid residues include aspartic acid and glutamic acid. The basic amino acid residue has a positive charge due to association with a H⁺ ion at physiological pH and the residue is attracted by aqueous solution so as to seek the surface positions in the conformation of a peptide in which it is contained when the peptide is in aqueous medium at physiological pH. Naturally occurring basic amino acid residues include the non-cyclic amino acids arginine, lysine, ornithine, diamino-butyric acid, and the cyclic amino acid histidine. The polar amino acid residue is not charged at a physiological pH, but the residue is not sufficiently repelled by aqueous solutions so that it would seek inner positions in the conformation of a peptide in which it is contained when the peptide is in aqueous medium. Naturally occurring polar amino acid residues include asparagine, glutamine, serine threonine, and cysteine in the reduced stage such as the SH-form. Preferably, the terminal amino acid at the C-terminus is modified to be a hydrophilic amino acid residue. Thus, for example, in PRGGSK (SEQ ID No. 60), the glycine at position 4 can be replaced by another amino acid residue to give the peptide PRGX_{bb}SK (SEQ ID No. 61), where X_{bb} can be a neutral, hydrophobic or charged residue such as Asn, Phe, Val, Asp, or Arg.

Also included with the scope of the present invention are analogues comprising amino acids that have been altered by chemical means such as methylation (e.g., α-methylvaline), amidation of the C-terminal amino acid by an alkylamine such as ethylamine, ethanolamine or ethylene diamine, and/or acylation or methylation of an amino acid side chain function (e.g., acylation of the epsilon amino group of lysine). Preferably, the C-terminal of the peptide is protected by amidation.

Cyclic Peptides

In one aspect of the invention, the peptide selected according to the criteria discussed above can be cyclic. Cyclic peptides may be prepared in which the ring is formed by oxidation of the naturally occurring cysteine residues yielding a disulfide bridged structure. For example, art known on-resin cyclization methods can be used to prepare cyclopeptides with bridges formed of thioethers, disulfides, or lactams between two side chains, lactams between the amino terminus and a side chain, and lactams between the amino and carboxy termini.

Typically, cyclic peptides are prepared using amino acids with orthogonally protected functional groups such that some protecting groups can be selectively removed in the presence of others. Those skilled in the art can use these techniques to prepare peptides in which the amino terminus is cyclized to the carboxyl terminus to form a ring. Alternatively, pairs of cysteine residues can be oxidized, in the solution or in solid phase, to disulfide bonds to form one or more rings, such as for forming cyclic hexapeptides.

In an alternate approach, cyclic peptides can be formed using side chain-to-side amide bonds or side chain-to-backbone linkages. Cyclic peptides cyclized in the head-to-tail fashion, have the advantage of having reduced number of conformational states available to them. This can often lead to more potent and/or more selective ligands to biological receptors or to tighter binding to antibody molecules. Further, the head to tail cyclic peptides are normally resistant to two of the three major types of proteolytic enzymes. Thus, neither aminopeptidases nor carboxypeptidases are activated since cyclization simultaneously removes both amino and carboxylate termini. The cyclic peptides can also have modified resistance to endopeptidases.

As one of skill in the art will recognize, the collection of peptides that tile across the sequence of LFA-1 or ICAM-1, that have different lengths, amino acid modifications and are linear or cyclic can form a library. The diversity of the library can be controlled by varying one or more of the factors above. The peptide library can be used in drug screening assays whereby lead compounds for drug development are identified.

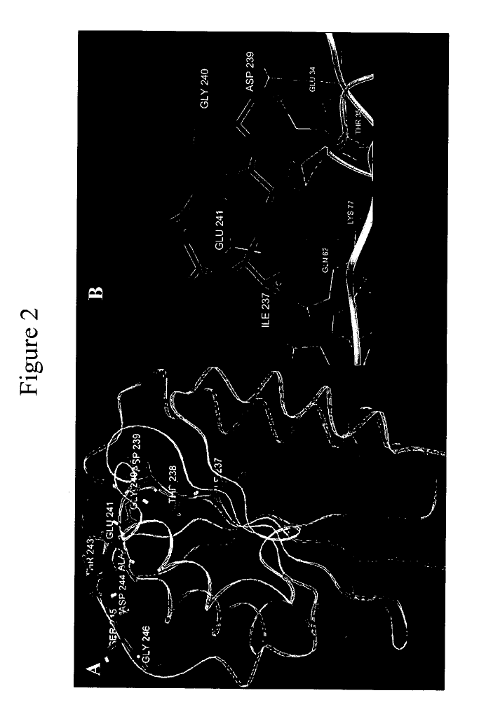

Peptides Docking to the D1 Domain of ICAM-1—The peptides cyclized as described above can be shown to have similar binding with the receptor proteins as the linear peptides. Any of the art known methods can be used. For example, AutoDock performs automated docking of the whole ligand with user-specified dihedral flexibility within a rigid protein binding-site. Typically, the program uses a Monte Carlo simulated annealing technique for configurational and translational exploration with a rapid energy evaluation and does not require subsequent energy minimization. The software applications include the following: (1) Insight II (BIOSYM Technologies) to generate missing hydrogen atoms of protein, (2) AutoDock (version 2.4) to dock the peptides to protein, and (3) RasMol (version 2.5) to calculate and examine the interactions between the docked peptide and the proteins. The coordinates of D1 domain of ICAM-1 can be obtained from the Brookhaven Protein Data Bank (PDB code IIC1); only the D1 domain (residues 1-83) was used as the target. The cyclic peptides can be built with the Biopolymer module of Insight II, the structures can be minimized, and the energy minimized structures can be subjected to AutoDock docking runs. For example, the linear 10 amino acid residues of LAB.L, ITDGEATDSG (Ile²³⁷-Gly²⁴⁶), were mapped onto ribbon of the I domain (FIG. 2A). The linear peptide localized in proximity of the divalent cation binding pocket on the upper face of the I-domain. The low-energy docked-model of the cyclic derivative of cLAB.L, cyclic-ITDGEA (Ile²³⁷-Ala²⁴², SEQ ID No. 60), to the D1 domain of ICAM-1 exhibits a docking energy of −52.97 kcal/mol (FIG. 2B). The backbone conformation of cyclic-ITDGEA was fixed while all the side chains were allowed to rotate freely. A grid of probe atom-interaction energies was computed on the basis of 37.5 Å side grids with a spacing of 0.375 Å. The ligands were then docked by simulated annealing with the the starting temperature selected to be 616 K. The lowest energy structure out of 100 docked structures, based on the force field scoring, was considered as the predicted binding conformation. The docked-model of cyclic-ITDGEA to D1 domain of ICAM-1 indicates the presence of extensive specific and non-specific interactions between them involving at least four residues on the cyclic-ITDGEA, and is similar to the binding of the corresponding linear peptide. Thus, cyclization of the peptides of the invention does not affect binding to the receptor proteins.

Thus, for example, VILPRG (SEQ ID No. 42), and PRGGSV (SEQ ID No. 45) can be cyclized to give cVILPRG (SEQ ID No. 62) and cPRGGSV (SEQ ID No. 63) respectively.

| | |
|---|---|
| SEQ ID No.62 | SEQ ID No.63 |

| | |
|---|---|
| Pro-Arg-Gly | Pro¹-Arg²-Gly³ |
| \| \| | \| \| |
| Leu-Ile-Val | Val⁶-Ser⁵-Gly⁴ |

Further, the peptides of SEQ ID No. 55-60, when cyclized, provide the following compounds of SEQ ID Nos. 64-69 respectively:

| Lys-Arg-Gly | Pro-Lys-Gly | Pro-Arg-Lys | Pro-Arg-Gly | Pro-Arg-Gly | Pro-Arg-Gly |
|---|---|---|---|---|---|
| \| \| | \| \| | \| \| | \| \| | \| \| | \| \| |
| Val-Ser-Gly | Val-Ser-Gly | Val-Ser-Gly | Val-Ser-Lys | Val-Lys-Gly | Lys-Ser-Gly |
| SEQ ID No.64 | 65 | 66 | 67 | 68 | 69 |

IV. Linkers

In one aspect of the invention, the peptides, either linear or cyclic, are attached to a linker group L. The linker L can be a direct bond, or a group having between 1 and 20 carbon atoms. Thus, for example, the linker (L) can be a straight or branched alkyl chain, such as, for example, propyl, butyl, octyl, and the like. Further, L can be a direct bond or a linking group having from 1 to 3 atoms independently selected from unsubstituted or substituted carbon, N, O or S. Representative linking groups useful in the compounds of the invention include, for example —O—, —S—, —NH—, —CH₂—, —OCH₂—, OC(O)—, —CO₂—, —NHC(O)—, —C(O)NH—, —OC(O)CH₂—, —OC(O)NH—, and —NHC(O)NH—, N(R₁)(CH₂)ₘ (wherein R₁ is substituted or unsubstituted aryl, heteroaryl, aralkyl, or heteroarylalkyl, and m is 0 or 1), (CH₂)N(R₁)(CH₂)ₘ, SO, SO₂, OCH₂, SCH₂, SOCH₂, SO₂CH₂, or CR₂R₃ (wherein R₂ and R₃ are independently selected from the group consisting of hydrogen, hydroxy, aryl, and heteroaryl).

In another aspect, L is a linking group defined by the formula:

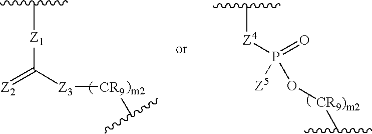

Z₁, Z₂, and Z₃ are independently selected from O, S, or NR₄, where R₄ is H or lower alkyl;

Z₄ is O or NH,

Z₅ is OR′, SR′, or methyl wherein R′ is selected from the group consisting of hydrogen, alkyl, aryl and salts thereof, and

R₉ is hydrogen, halogen, or alkyl.

In yet another aspect, the linking group L can be amino acid residues. Amino acid linkers are usually at least one residue and can be 40 or more residues, but preferably about 1 to 10 amino acid residues in length. Typical amino acid residues used for linking are tyrosine, cysteine, lysine, glutamic and aspartic acid, or the like.

V. The Moiety

The compounds of the invention include a moiety covalently linked to the peptide via a linker. The moiety includes intercalators, reporter molecules, dyes, and drugs, and includes toxins, cytotoxins, alkylating agents, enzymes, enzyme inhibitors, sequences of RNA or DNA intended for cellular transcription or anti-sense inhibition, antibiotics, antimetabolites, hormones, neurotransmitters, radioopaque dyes, radioactive isotopes, magnetic spin resonance agents, fluorogenics, bio-markers, lectins, photochemicals, cell membrane modifiers, antiproliferatives and heavy metals. Typical intercalators, reporter molecules, and dyes include fluoresceins, rhodmines, coumarins, acridines, xanthenes, antraquinones, and the like. Suitable fluorescent compounds include, but are not limited to, fluorescein, 5-carboxyfluorescein (FAM), fluorescein iso-thiocyanate (FITC), rhodamine, 5-(2′-aminoethyl) aminonapthalene-1-sulfonic acid (EDANS), anthranilamide, coumarin, terbium chelate derivatives, Reactive Red 4, BODIPY dyes and cyanine dyes, Alexa 488, Cy3, Cy5, PE, Texas Red, Cascade Blue, Bodipy, and tetramethyl rhodamine isothiocyanate (TRITC). Preferred fluorescent labels are fluorescein (5-carboxyfluorescein-N-hydroxysuccinimide ester), rhodamine (5,6-tetramethyl rhodamine), substituted rhodamine compounds, and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 mn), thus allowing their simultaneous detection. The fluorescent labels can be obtained from a variety of commercial sources, including Molecular Probes, Eugene, Oreg. and Research Organics, Cleveland, Ohio.

Other detectable labels include molecular or metal barcodes, mass labels, and labels detectable by nuclear magnetic resonance, electron paramagnetic resonance, surface-enhanced raman scattering, surface plasmon resonance, resonance raman, microwave, or a combination thereof. Mass labels are compounds or moieties that have, or which give the labeled component, a distinctive mass signature in mass spectroscopy. Mass labels can be useful when mass spectroscopy is used for detection. Combinations of labels can also be useful. In some applications, metal barcodes can be used as the detectable label. Metal barcodes are 30-300 nm in diameter by 400-4000 nm multilayer multi-metal rods. These rods are normally constructed by electrodeposition into an alumina mold, then the alumina is removed to obtain the multilayered metal barcodes. The metal barcodes can have up to 12 zones encoded, in up to 7 different metals, where the metals have different reflectivity and thus appear lighter or darker in an optical microscope depending on the metal, thereby providing the identification codes.

In another aspect, the moiety covalently linked to the peptide via a linker can be a drug for use in the treatment of cancer. The cancer can be any type of cancer, such as for example, a breast cancer, an ovarian cancer or a gastrointestinal cancer includes gastric cancer, small bowel cancer, colon cancer, and rectal cancer. The cancer can further include lymphoma, adenocarcinoma, glioblastoma, leukemia, esophageal carcinoma, head and neck cancer, prostate cancer, lung cancer, melanoma, cervical carcinoma, pancreatic cancer, sarcoma, hepatoma, and gallbladder cancer. The drug can be, for example, methotrexate, mitomycin C, carboplatin, cisplatin, paclitaxel, etoposide, or doxorubicin. Thus, the drug can be an alkylating agent such as cyclophosphamide, isosfamide, melphalan, hexamethylmelamine, thiotepa, dacarbazine, carmustine (BSNU) or lomustine (CCNU); an antimetabolite such as pyrimidine analogues, for instance 5-fluorouracil and cytarabine or its analogues such as 2-fluorodeoxycytidine; a folic acid analogue such as methotrexate, idatrexate or trimetrexate; a spindle poison including vinca alkaloids such as vinblastine or vincristine or their synthetic analogues such as navelbine, or estramustine; a taxoid; an epidophylloptoxin such as etoposide or teniposide; an antibiotic such as danorubicine, doxorubicin, bleomycin or a mitomycin; a topoisomerase inhibitor such as camptothecin derivatives chosen from CPT-11 and topotecan or pyridobenzoindole derivatives, and various agents such as procarbazine, mitoxantrone, platinum coordination complexes such as cisplatin or carboplatin, a telomerase inhibitor such as GRN 163, and biological response modifiers or growth factor inhibitors such as interferons or interleukins. Thus, the moiety can be doxorubicin, vinblastin, methotrexate, retinoids, and carotenoids.

In another aspect of the invention, the moiety covalently linked to the peptide via a linker can be a drug for use in the treatment of rheumatoid arthritis (RA). RA is a debilitating, chronic inflammatory disease affecting 1 to 2% of the world's population. This condition causes pain, swelling and destruction of multiple joints in the body and can also result in damage to other organs such as the lungs and kidneys. Recent recommendations of the American College of Rheumatology include early initiation of disease-modifying anti-rheumatic drug (DMARD) therapy for any patient with an established diagnosis and ongoing symptoms. Anticancer drugs have become the first line therapy for the vast majority of patients, with the chemotherapeutic drug, methotrexate, being the drug of choice for 60 to 70% of rheumatologists. The severity of the disease often warrants indefinite weekly treatment with this drug and, in those patients whose disease progresses despite methotrexate therapy (over 50% of patients), second line chemotherapeutic drugs such as cyclosporin and azathioprine (alone or in combination) are frequently employed. Thus, the drugs for conjugation to peptides for the treatment of RA includes the drugs for use in cancer therapy.

In yet another aspect, the moiety covalently linked to the peptide via a linker can be a drug for use in the treatment of multiple sclerosis (MS). MS is a common chronic inflammatory disease involving the nervous system. Typically, in MS recurring episodes of adverse neurological deficits occur over a period of several years, with relatively stable periods between the episodes. Roughly half of MS cases progress to a more chronic phase. Typically, the disease cripples the patient by disturbing visual acuity; stimulating double vision; disturbing motor functions affecting walking and use of the hands; producing bowel and bladder incontinence; spasticity; and sensory deficits (touch, pain and temperature sensitivity). Drugs for MS include methotrexate, cyclosporin, azathioprine, interferon-β, Betaseron™, Avonex™, leflunomide, and the like.

In another aspect, the moiety covalently linked to the peptide via a linker can be a drug for use in the treatment of psoriasis. Psoriasis is a common, chronic inflammatory skin disease characterized by raised, inflamed, thickened and scaly lesions, which itch, bum, sting and bleed easily. In approximately 10% of patients, psoriasis is accompanied by pronounced arthropathic symptoms that are similar to the changes seen in rheumatoid arthritis. Approximately 2 to 3% of the U.S. population suffers from psoriasis, with 250,000 new cases being diagnosed each year. Drugs for conjugation for the treatment of psoriasis includes steroids, ultra violet B, PUVA, methotrexate, leflunomide, and cyclosporine, and their active metabolites.

In another aspect, the moiety covalently linked to the peptide via a linker can be a drug for use in the treatment of HIV infection. The anti-HIV drug can be a commercially available drug, such as, for example, a nucleoside analog which includes Zidovudine™, Didanosine™, Zalcitabine™, Stavudine™, Lamivudine™, and Viread™; a protease inhibitor which includes Indinavir™, Nelfinavir™, Saquinavir™ and Ritonavir™; a non-nucleoside reverse transcriptase inhibitors (NNRTI) which include Nevirapine™, Delavirdine™ and Efavirenz™; and a HIV-fusion inhibitor, such as Fuzeon™. The anti-HIV drug can also be experimental drugs, such as, for example, T-1249, or other compounds known in the art.

Thus, the present invention provides compositions and methods for the treatment or prevention of an autoimmune disorder affecting any body cell, tissue, organ or organ system, including but not limited to cutaneous, cardiac, pericardial, endocardial, vascular lining or wall, blood, blood-forming (e.g., marrow or spleen), endocrine (e.g., pancreatic or thyroid), gastrointestinal (e.g., bowel), respiratory (e.g., lung), renal, central nervous system, peripheral nervous system, muscular or skeletal joint (e.g., articular cartilage or synovial) tissue. The methods and compositions of the present invention can, therefore, be utilized to treat any autoimmune disorder including, but not limited to atopic dermatitis, contact dermatitis, eczematous dermatitides, seborrheic dermatitis, Lichen planus, pemphilgus, bullous pemphigus, Epidermolysis bullosa, Alopecia areata, urticaria, angioedemas, erythema, eosinophilias, migraine, lupus, including cutaneous lupus (discoid lupus erythematosus), extracutaneous lupus, including systemic lupus erythematosus, acute lupus, lupus annularis, lupus discretus, lupus lymphaticus, lupus papillomatis, lupus psoriasis, lupus vulgaris, lupus sclerosis, neonatal lupus erythematosus, and drug-induced lupus; anti-phospholipid syndrome (APS), hemolytic anemia (HA), idiopathic thrombocytopenia (ITP), thyroiditis, diabetes mellitus (DM), inflammatory bowel disease, e.g., Crohn's disease or ulcerative cholitis, rhinitis, uveitis, nephrotic syndrome, demyelinating diseases such as multiple sclerosis (MS), myasthenia gravis (MG), and arthritis, e.g., rheumatoid arthritis, psoriac arthritis, non-rheumatoid inflammatory arthritis, arthritis associated with Lyme disease, or osteoarthritis.

Thus, for example, PRGGSV (SEQ ID No. 45) can be cyclized to cPRGGSV (SEQ ID No. 63), the serine residue at position 5 can be replaced by a lysine (SEQ ID No. 68), and conjugated to methotrexate (MTX) to give compound (I) below.

| | |
|---|---|
| Pro-Arg-Gly | (I) |
| \| \| | |
| Val-Lys-Gly | |
| \| | |
| MTX | |

Similarly, the cyclic peptides of SEQ ID Nos. 64-69 can be conjugated to a linker L via a lysine group, and then a moiety, such as MTX can be attached to the linker. Shown below are compounds II-VI that are derived from the cyclization of peptide PRGX_{bb}SK (SEQ ID No. 61), where X_{bb} can be a neutral, hydrophobic or charged residue such as Asn, Phe, Val, Asp, or Arg.

| | | | | |
|---|---|---|---|---|
| Pro-Arg-Gly | Pro-Arg-Gly | Pro-Arg-Gly | Pro-Arg-Gly | Pro-Arg-Gly |
| \| \| | \| \| | \| \| | \| \| | \| \| |
| Lys-Ser-Asn | Lys-Ser-Phe | Lys-Ser-Val | Lys-Ser-Asp | Lys-Ser-Arg |
| L | L | L | L | L |
| MTX | MTX | MTX | MTX | MTX |
| (II) | (III) | (IV) | (V) | (VI) |

The linker (L) can be a direct bond, or a group having between 1 and 20 carbon atoms, as explained in detail above. In the hexapeptides above, the formation of the cyclic ring stabilizes the β-turn around the Pro-Arg-Gly sequence which can be important for binding to the LFA-1 receptor. However, any of the amino acids can be replaced. For example, the glycine residue at position 3 of SEQ ID No. 45 can be X_{bb} to give compounds of formula VII below:

| | |
|---|---|
| Pro-Arg-Xbb | (VII) |
| \| \| | |
| Lys-Ser-Gly | |
| L | |
| MTX | |

where X_{bb} can be a neutral, hydrophobic or charged residue such as Asn, Phe, Val, Asp, or Arg, and the linker (L) can be a direct bond, or a group having between 1 and 20 carbon atoms. As one of skill will realize, cyclic peptides of 4, 5, 6, 7, 8, 9, 10, 11, or 12 amino acids, selected from the sequence of LFA-1 or ICAM-1, can be prepared, and conjugated to a moiety via a linker.

IV. Synthesis of the Peptides

The composition and methods of the invention comprise peptides, as described above. The peptides of the present invention can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTY 3^{rd} Ed., Vols. A and B (Plenum 1992), and Green and Wuts, PROTECTIVE GROUPS IN ORGANIC SYNTHESIS 2^{nd} Ed. (Wiley 1991). Starting materials for the compounds of the invention may be obtained using standard techniques and commercially available precursor materials, such as those available from Aldrich Chemical Co. (Milwaukee, Wis.), Sigma Chemical Co. (St. Louis, Mo.), Lancaster Synthesis (Windham, N.H.), Apin Chemicals, Ltd. (New Brunswick, N.J.), Ryan Scientific (Columbia, S.C.), Maybridge (Cornwall, England) and Trans World Chemicals (Rockville, Md.).

The procedures described herein for synthesizing the compounds of the invention may include one or more steps of protection and deprotection (e.g., the formation and removal of acetal groups). In addition, the synthetic procedures disclosed below can include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. Methods of protection and deprotection, purification and identification and quantification are well known in the chemical arts.

Synthesis of Linear Peptides—The solid phase syntheses of linear peptides can be carried out using Pioneer Peptide Synthesis System (PerSeptive Biosystems), in which peptide chains can be assembled on a solid support from the C-terminal of one amino acid at a time and elongating the chain toward the N-terminal. The peptide can be cleaved from the support to allow isolation of the final product. The Pioneer Peptide Synthesis System automates the 9-fluorenylmethoxycarbonyl (Fmoc) method of peptide synthesis, by which Na-amino group of each amino acid is temporarily protected by the Fmoc group. The Fmoc group can be rapidly removed using a base in an organic solvent, such as 20% piperidine in DMF. Typically, the solid support can be Fmoc-PAL-PEG-PS and the PAL linker can be [5-(4-Fmoc-aminomethyl-3,5-dimetoxyphenoxy) valeric acid]. PEG-PS support can be prepared from long polyethylene-glycol molecules grafted onto polystyrene. The activation of amino acids can be achieved by using the activator in the form of N-[(Dimetilamino)-1H-1,2,3-triazolo[4,5-b]pyridin-1-ylmethylene]N-methylmethanaminium Hexafluorophosphate N-oxide (HATU) in the presence of N,N-diisopropylethyl amine (DIEA), the solvent was N,N-dimethyl formamide (DMF). Cleavage from the resin and deprotection of the peptide can be achieved with 2,2,2-trifluoroacetic acid (TFA) containing water in a 95:5 ratio at the room temperature for about 1 hour. The cleavage cocktail with support can be purified by precipitating the peptides by adding an organic solvent, separating the peptides by centrifugation, and drying by lyophilization. The purity and molecular weight of the individual peptide can be determined by analytical HPLC and FABMS, or any other analytical technique.

Synthesis of Cyclic Peptide—The cyclization of the linear peptides can be accomplished by the standard high-dilution technique using benzotriazolyloxytetramethylivonium hexafluorophosphate (HBTU) in the presence of NMM in DMF as solvent to give cyclic peptide. Hydrogenolysis of the cyclic peptide to remove the Bzl protecting groups from Thr, Asp and Glu can be achieved with 10% of palladium on activated carbon (Pd/C). as a catalyst under an H₂ atmosphere in EtOH to yield the desired product. The crude product can be purified by preparative reversed-phase HPLC and analyzed by analytical reversed-phase HPLC and MS. A typical synthesis of a cyclic peptide is given below:

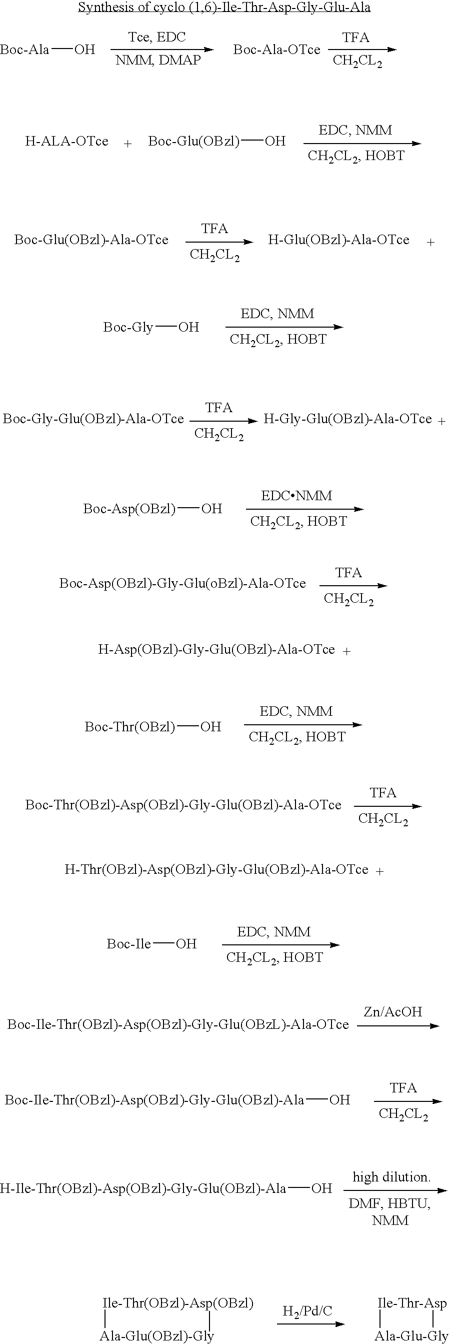

Synthesis of P-L-M—The methotrexate-ICAM-1 peptide conjugates were synthesized by conjugating the γ-carboxylic acid of glutamic acid in methotrexate (MTX) to the N-terminus of the ICAM-1 peptides (Scheme 2).

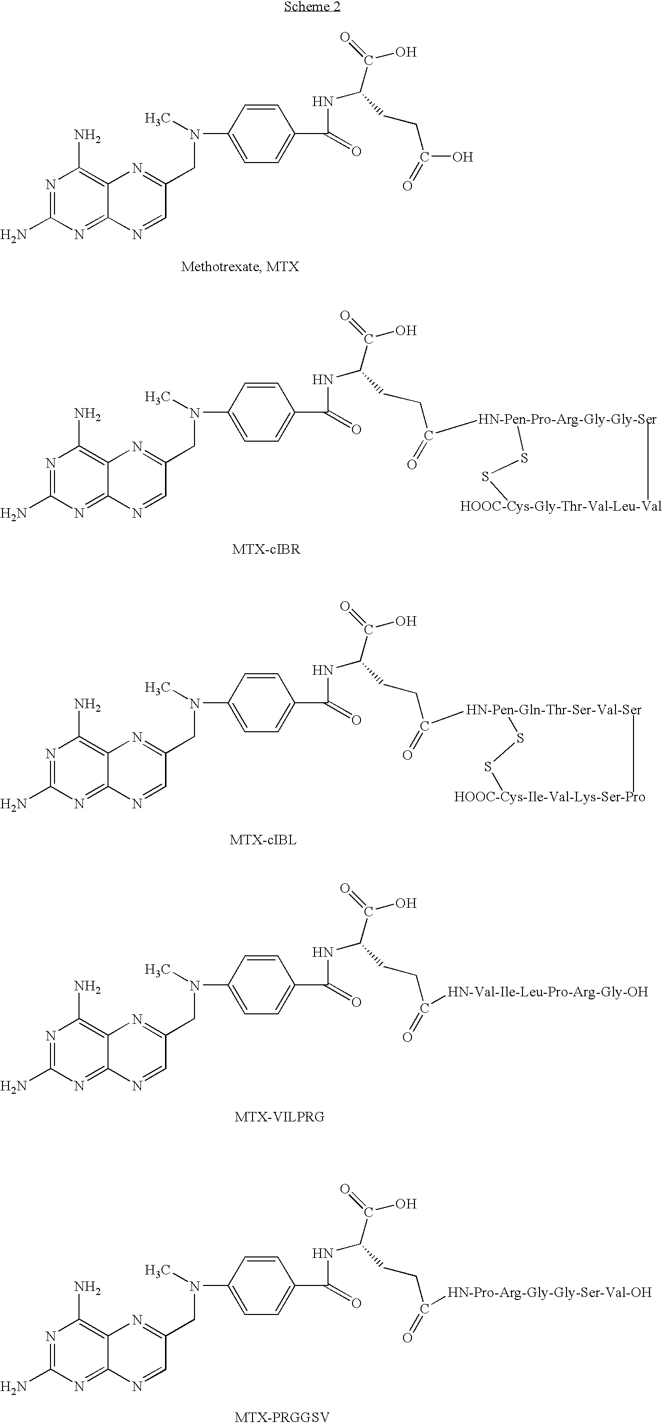

Methotrexate (MTX) with a protected α-carboxylic acid can initially be synthesized. The carboxylic acid group in MTX can be activated with benzotriazolyloxytetramethyl-ivonium hexafluorophosphate (HBTU) in the presence of an amine, such as N,N-diisopropylethyl amine (DIEA) in an organic solvent, such as N,N-dimethyl formamide (DMF), followed by the reaction with amine group of Glu(O-tBu)-OH to give a selectively protected MTX (MTX-(OtBu)). Next, a solution of peptide is added dropwise to a solution of HBTU, MTX-(OtBu), and DIEA in an organic solvent. The tert-butyl protecting group in the Glu γ-carboxylic acid can be removed by treatment with an acid, such as trifluoroacetic acid in dichloromethane. The crude product can be purified, such as, by semi-preparative HPLC using a C-18 column to give MTX-peptide conjugates. The synthesis of cLAB.L-MTX conjugate is given below:

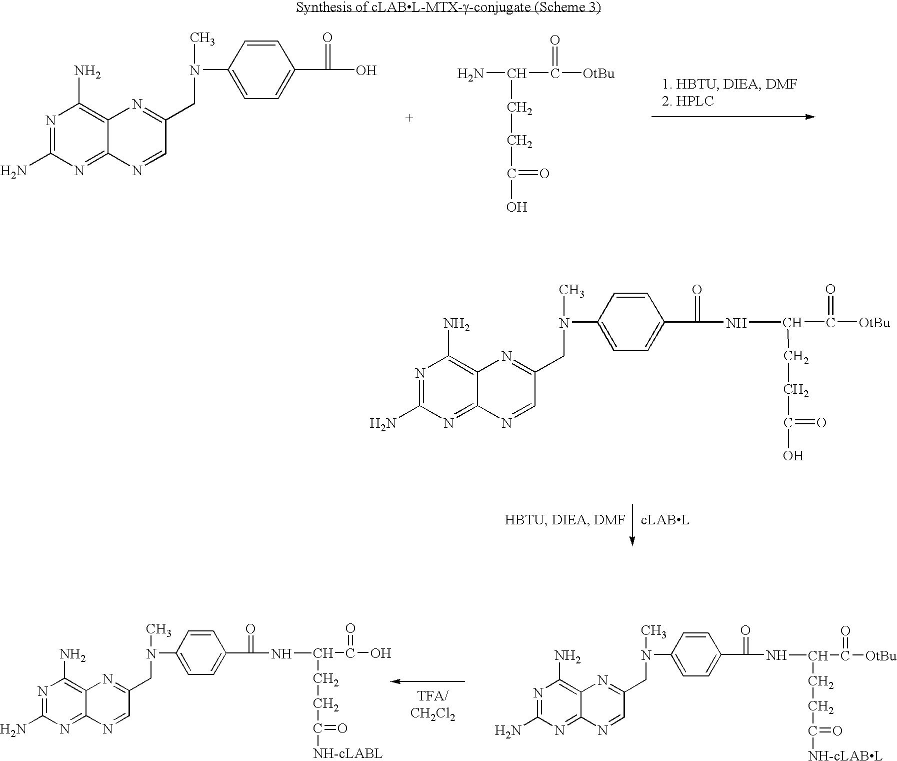

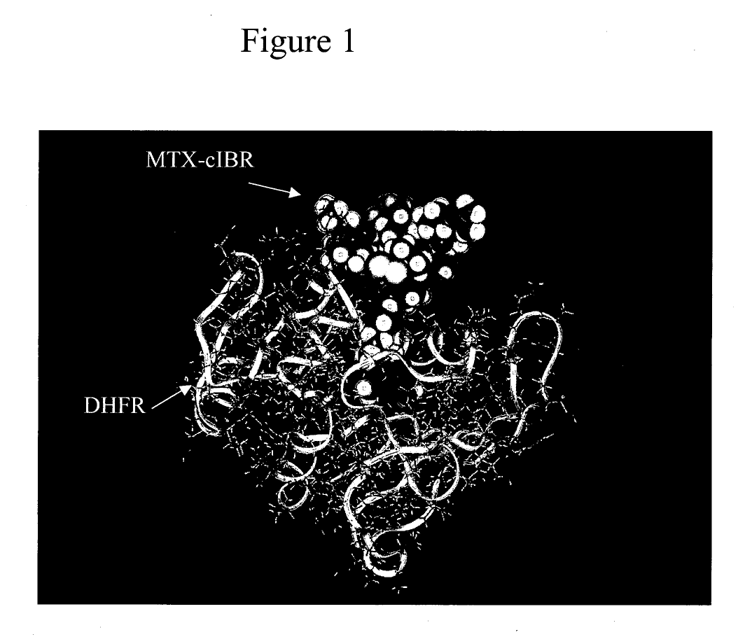

P-L-M Docking—The MTX-cIBR conjugate structure created by InsightII was overlaid with the crystal structure of MTX bound to the active site of DHFR to determine if any obvious steric hindrances arise upon MTX conjugation (FIG. 1). The crystal structure of MTX bound to DHFR was obtained from the Protein Data Bank (PDB; PDB code: 1DF7). Only one potential hindrance was detected, which was resolved by rotation of the solvent-exposed Arg-57 of DHFR. This model suggests that the α-carboxylic acid group of MTX may then form a salt bridge with the basic side chain of Arg-57 in DHFR. Additionally, the position of the pteridine ring and p-aminobenzoyl moiety of MTX relative to DHFR residues are similar to those previously studied by X-ray crystallography and NMR. The pteridine ring fits into a hydrophobic pocket created by Ile-5, Ala-6, Leu-27, and Phe-30. The p-aminobenzoyl moiety lies in a neighboring pocket surrounded by the lipophilic side-chains of Ala-6, Leu-27, and Phe-30 (on one side) and of Phe-49, Pro-50, and Leu-54 (on the other). In addition, this model also suggested that DHFR residues (Arg-52, Pro-53, and Lys-32) could form a pocket into which cIBR peptide residues (Thr-10, Gly-11, and Ser-6) may be inserted and interact. The docking showed that the P-L-M compounds of the invention can bind to the receptor sites, and the conjugation of the peptides with moieties, such as the drug MTX, does not adversely affect the binding.

V. Pharmaceutical Formulations and Modes of Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sufobutyl ether cyclodextrins) etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, compounds of this invention will be administered as pharmaceutical formulations including those suitable for oral (including buccal and sub-lingual), rectal, nasal, topical, pulmonary, vaginal or parenteral (including intramuscular, intraarterial, intrathecal, subcutaneous and intravenous) administration or in a form suitable for administration by inhalation or insufflation. The preferred manner of administration is intravenous using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents, tonicifying agents, and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

For oral administration, the composition will generally take the form of a tablet, capsule, a softgel capsule or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils, fatty esters or polyols are conventionally employed as solvents or suspending media. In addition, parenteral administration may involve the use of a slow release or sustained release system such that a constant level of dosage is maintained.

Alternatively, the pharmaceutical compositions of the invention may be administered in the form of suppositories for rectal or vaginal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the invention may also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or gel reservoir, or may take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.05 mg/kg to about 40 mg/kg body weight, more preferably about 0.5 mg/kg to about 20 mg/kg, in at least one dose. In larger mammals the indicated daily dosage can be from about 1 mg to 100 mg, one or more times per day, more preferably in the range of about 10 mg to 50 mg. The subject may be administered as many doses as is required to reduce and/or alleviate the signs, symptoms, or causes of the disorder in question, or bring about any other desired alteration of a biological system. One of ordinary skill in the art of treating such diseases will be able, without undue experimentation and in reliance upon personal knowledge and the disclosure of this application, to ascertain a therapeutically effective amount of the compounds of this invention for a given disease.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

As discussed above, the pharmaceutical formulations may contain one or more of the conjugates described above and additionally one or more active agents that effectively provide treatment for the subject. The additional active agent may be, but is not limited to, a 5-HT3 antagonist or agonist, a GABA antagonist or an agonist, a NSAID, 5-HT1A ligand, sigma receptor ligand, a COX-2 inhibitor, or another pain killer, a steroid, a vitamin, or a hormone, and combinations thereof. This additional active agent can be administered to the subject prior to, concurrently with or subsequently to administration of the compositions of this invention. Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention.

VI. Kits

In another aspect, the invention relates to pharmaceutical compositions in kit form. The kit comprises container means for containing the compositions such as a bottle, a foil packet, or another type of container. Typically the kit further comprises directions for the administration of the compositions. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be administered. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent, such as, for example, a mechanical counter which indicates the number of daily doses that has been dispensed, a microchip memory coupled with a liquid crystal readout, or audible reminder signal which, for example, reads out the date that the last daily dose has been taken and/or reminds one when the next dose is to be taken, and the like.

### EXAMPLES

Below are examples of specific embodiments for carrying out the present invention. The examples are offered for illustrative purposes only, and are not intended to limit the scope of the present invention in anyway. Efforts have been made to ensure accuracy with respect to numbers used (e.g., amounts, temperatures, etc.), but some experimental error and deviation should, of course, be allowed for. Cyclic peptides cIBL and cIBR were purchased from Multiple Peptide System (San Diego, Calif.). The following materials were purchased from Sigma (St. Louis, Mo.) or (Dorset, UK): dihydrofolate reductase enzyme (DHFR), β-NADPH, dihydrofolic acid, RPMI 1640 medium (containing NaHCO₃ and D-glucose), dialyzed (DFCS) and non-dialyzed fetal calf serum (NFCS), RNase A, deoxyuridine (dUrd), propidium iodide (PI), dextran, perchloric acid, and activated charcoal. [5-³H]-dUrd was purchased from Moravek Biochemicals (Brea, Calif.). Gentamicin, amphotericin B and L-glutamine were purchased from Gibco BRL (Paisely, Scotland). FITC-labeled monoclonal anti-human antibody CD11a (clone 38) was purchased from Ancell (Bayport, Minn.).

### Example 1

Cell Cultures

Molt-3, Caco-2 and Calu-3 cell lines were obtained from the American Type Culture Collection (Rockville, Md.). Molt-3 and Caco-2 cells were maintained and grown using known methods. Briefly, the Caco-2 cell-line was grown as monolayers in Dulbecco's modified Eagle's medium (DMEM) with 25 mM glucose containing 10% FBS, 1% nonessential amino acids, 1 mM Na-pyruvate, 1% L-glutamine and 100 μg/l of penicillin/streptomycin. Cells were grown in 75-cm² tissue culture flasks (Falcon) for maintenance purposes and in a 48-well cell culture cluster (Costar) for heterotypic-adhesion experiments. Before confluency was reached, Caco-2 cells were induced with 100 U/mL IFN-γ for 24 h to up-regulate the ICAM-1 expression. Calu-3, a lung epithelial cell line, was maintained in a 1:1 mixture of Ham's F12:DMEM containing 10% FBS and 100 μg/mL penicillin/streptomycin. Upon reaching 90% confluency (approximately 4-5 days), cells were subcultured at a 1:2 split ratio using 0.25% trypsin/0.1% EDTA. Calu-3 cells were induced with 500 U/mL IFN-γ for 48 h to up-regulate the ICAM-1 expression. All cell lines were grown in a 95% humidified/5% CO₂ atmosphere at 37° C.

Molt-3 Cells: MOLT-3 cells, a leukemia-derived human T-cell line, were purchased from ATCC (Rockville, Md.). These cells were propagated in RPMI-1640 medium (Sigma) containing 10% v/v fetal bovine serum and penicillin/streptomycin (100 mg/L medium) and incubated at 37° C. with 95% humidity and 5% CO₂. L1210-WT and L1210-1565 mouse leukemia cell lines were obtained from the Institute for Cancer Research (UK) and cultured in RPMI 1640 medium (containing NaHCO₃ and D-glucose). RPMI medium (500 mL) was supplemented with 50 mL of either dialyzed (DFCS) or non-dialyzed fetal calf serum (NFCS). In addition, supplements of 0.2 mL of 50 μg/mL gentamicin, 1 mL of 250 μg/mL amphotericin B, and 5 mL of 200 mM L-glutamine were also added.

Human peripheral blood lymphocytes (PBL): Human PBL were isolated from whole blood drawn in sodium citrate tubes immediately before use. Red blood cells (RBC) were lysed using a Leinco Easy-Lyse Kit (#K104) and PBLs further purified by leukocyte separation media (LSM) gradient. Briefly, 2 mL of LSM was added to 8 mL of cells suspended in buffer and spun for 20 min at 1600 rpm. Five mL of the supernatant was removed and the next 4 mL was removed and diluted with PBS, 5% FBS. These cells were then spun for 10 min at 1600 rpm, washed with PBS at 4° C., and diluted to the desired concentration.

Human KB epithelial l cells: The human KB epithelial cell line was a gift from Dr Gerrit Jansen (University Free Hospital, Amsterdam, Netherlands) and was developed to overexpress the membrane folate binding protein (mFBP). Cells were cultured in RPMI 1640 medium without folic acid and supplemented with 10% heat inactivated dialyzed FCS (50 mL volume), 0.2 mL of 50 μg/mL gentamicin, 1 mL of 50 μg/mL amphotericin B, and 5 mL of 200 mM L-glutamine. The folate source, 20 nM (R,S)-LV, was added to supply cells with adequate growth conditions.

### Example 2

Synthesis of Linear Peptides.

Solid phase syntheses of linear peptides (VILPRG (SEQ ID No. 42) and PRGGSV (SEQ ID No. 45)) derived from ICAM-1 protein were carried out using the 9-fluorenyl-methoxycarbonyl (Fmoc) method by an automated Pioneer Synthesis System (PerSeptive Biosystems). Fmoc-PAL-PEG-PS solid support was used to synthesize the peptides. The amino acids were activated with N-[(dimethylamino)-1H-1,2,3-triazolo [4,5-b]pyridine-1-ylmethylene]N-methyluronium hexafluorophosphate N-oxide (HATU) in the presence of N,N-diisopropylethyl amine (DIEA) in N,N-dimethyl formamide (DMF). The peptide was cleaved from the resin using trifluoro acetic acid (TFA), precipitated in diethyl ether, and isolated by centrifugation or filtration. The crude product was purified with a semi-preparative C-18 column (12 μm, 300 Å, 25 cm×21.4 mm i.d., flow rate 10 mL/min) using HPLC with acetonitrile and 0.1% TFA in water as solvents. The pure fractions were collected and dried by lyophilization. The purity and molecular weight of each peptide was determined by analytical HPLC (5 μm, 300 Å, 25 cm×4.6 mm i.d., flow rate 1 mL/min) and FAB.

### Example 3

Synthesis of Cyclic Peptides.

The synthesis of cyclic-ITDGEA (SEQ ID No. 60) derived from LFA-1 protein, consists of two steps. First is the synthesis of linear hexapeptide ITDGEA (SEQ ID No. 5) using the solution-phase Boc-amino acid chemistry, and the second part is reaction of cyclization by linking the N-terminal amino group of the Ile residue and C-terminal acido group of the Ala residue. The two-part procedure is finalized by the removal of side protection groups of the cyclic peptide. The synthesis of the linear peptide was initiated from amino acid Boc-Ala-OH. Trichloroethyl (Tce) ester was used as protecting group for the α-carboxyl group of Ala residue; Tce ester is quite stable to acidic conditions and can be removed by zinc in acetic acid (AcOH). Treatment of Boc-Ala-OH with 2,2,2-trichloroethanol in the presence of 1-[3-(dimethylamino)-propyl]-3-ethilcarbodiimide hydrochloride (EDC), 4-dimethylaminopiridine (DMAP), 1-hydroxybenzotriasole (HOBT), and N-methymorpholine (NMM) in the methylene chloride (CH₂Cl₂) as the solvents, yielded in the formation of Boc-Ala-OTce. This compound was then extracted with ethyl acetate (EtOAc), washed with saturated aqueous NaHCO₃ and NaCl, and dried over anhydrous Na₂SO₄ overnight. After solvent evaporation, the residue was triturated and washed with anhydrous ethyl ether (Et₂O) to give white solid at 78% recovery. The solid was isolated by decantation, dried under vacuum to remove residual Et₂O and used in the next step without further purification. The solid was assessed by analytical HPLC and FABMS. The formation of linear hexapeptide peptide was proceeded by series of coupling reaction of each amino acid. The standard solution-phase Boc-amino acid chemistry with EDC and HOBT was used as coupling reagents in the presence of NMM in the CH₂Cl₂ as solvents. The removal of Boc protection group was achieved by treatment with TFA and CH₂Cl₂ in a 1:1 ratio. The side chain of Thr, Asp and Glu were protected with benzyl protection groups (Bzl). Each residue after every coupling reaction was extracted with EtOAc, and washed sequentially with citric acid, saturated aqueous NaHCO₃ and NaCl before drying overnight over anhydrous Na₂SO₄. The coupling reaction yielded in 70-80% residue. Each linear residue was confirmed by analytical HPLC and FABMS. The cyclization of the linear hexapeptide was accomplished by the standard high-dilution technique using benzotriazolyloxytetramethylivonium hexafluorophosphate (HBTU) in the presence of NMM in DMF as solvent to give cyclic peptide in 45% yield after HPLC purification. Hydrogenolysis of the cyclic peptide to remove the Bzl protecting groups from Thr, Asp and Glu was achieved with 10% of palladium on activated carbon (Pd/C) as a catalyst under an H₂ atmosphere in EtOH to yield the desired product in the quantitative yield. The crude product was purified by preparative reversed-phase HPLC and analyzed by analytical reversed-phase HPLC and MS.

### Example 4

Synthesis of Methotrexate-Peptide Conjugates

Synthesis of MTX-peptide conjugates was accomplished by forming an amide bond between the N-terminus of the peptide and the y-carboxylic acid of MTX. The synthesis of MTX with a protected α-carboxylic acid was initiated by adding a solution of L-Glu(OH)-OtBu (0.132 mmol) in 5 mL of DMF dropwise into a mixture of 4-[N-(2,4-diamino-6-pteridinylmethyl)-N-methylamino] benzoic acid hemihydrochloride dihydrate (0.132 mM), HBTU, and DIEA in 5 mL of DMF. The reaction mixture was stirred 2 h at room temperature under nitrogen. The crude product was concentrated under reduced pressure to yield MTX-(OtBu) as a yellow oil. The product was further purified by preparative HPLC to give 87% yield. Mass spectroscopy (FAB) analysis indicated the product had the expected MW of 511 (M+1).

Next, a solution of peptide (0.098 mmol) was added dropwise to a solution of HBTU, MTX-(OtBu), and DIEA (0.098: 0.098: 0.198 mmol) in 5 mL of DMF. The mixture was stirred for 3 h at room temperature under nitrogen. The reaction was concentrated under reduced pressure to give an oily residue. The resulting residue was dissolved in 3 mL of CH₂Cl₂ followed by addition of 3 mL of TFA. After the solution was stirred for 1 h at room temperature, the solvents were evaporated and the crude product purified by semi-preparative HPLC using a C-18 column to give MTX-peptide conjugates in 60-70% yield. The final products were analyzed by analytical HPLC and MS (ESI).

Synthesis of Methotrexate-LFA-1 derived peptide Conjugates—Cyclic peptides cLAB.L (cyclol,12-PenITDGEATDSGC) and cLBE.L (cyclol,12-PenDLSTSLDDLRC) were purchased from Multiple Peptide Systems (San Diego, Calif.). The pure products were analyzed by NMR and fast atom bombardment mass spectrometry (FABMS). Synthesis of the MTX-cLAB.L was based on the formation of an amide bond between the N-terminal of cLAB.L and the carboxylic acid of methotrexate (MTX) following series of reactions described above in Scheme 3. The carboxylic acid group in compound 1 was initially activated with benzotriazolyloxytetramethylivonium hexafluorophosphate (HBTU) in the presence of N,N-diisopropylethyl amine (DIEA) in N,N-dimethyl formamide (DMF), and followed by the reaction with amine group of Glu(O-tBu)-OH (compound 2) to give a selectively protected α-carboxylic acid MTX (compound 3). The yield of this reaction was 85-92% after purification by preparative HPLC. The Glu γ-carboxylic acid in compound 3 was treated with HBTU and DIEA in DMF, and reacted with cLAB.L peptide to give MTX-cLAB.L (compound 4). The tert-butyl protecting groups (t-Bu) in the Glu γ-carboxylic acid of compound 4 was then removed by TFA in methylene chloride (CH₂Cl₂) in a 1:1 ratio for 45 min. The final conjugate was confirmed using FABMS (M+1=1634). The methodology for MTX-cLBE.L is similar to the synthesis of MTX-cLAB.L, the final conjugate of MTX-cLBE.L was also confirmed by FABMS (M+1=1428).

### Example 5

Heterotypic Adhesion Experiments with LFA-1 derived peptides—Two heterotypic cell adhesion systems were used in this work. The adhesion between Molt-3 T-cells/Calu-3 lung epithelial monolayers was used to assess the inhibitory activities of cLAB.L and cLAB.2L; Molt-3 T-cells/Caco-2 colon epithelial monolayers system was for cLAB.L and its derivatives. Briefly, Calu-3 or Caco-2 cell monolayers were pretreated with peptide solution prior to the adherence of fluorescence-labeled Molt-3 cells. Peptide was dissolved in RPMI-HEPES and added at various concentrations to the monolayers. In the case of Molt-3/Caco-2 adhesion, the inhibitory activity of a monoclonal antibody (mAb) to ICAM-1 (11C81, R&D Systems, Minneapolis, Minn.) was also tested. Since cLAB.L has been shown to bind to ICAM-1 on T-cells, irrelevant peptides with no activities on cell adhesion mediated by ICAM-1/LFA-1 interaction, were used as negative controls. Peptides or mAb was allowed to react with the cell surface receptors for 30 min at 4° C., followed by extensive washing with RPMI-HEPES and the adherence of labeled Molt-3 cells. Activated Molt-3 cells were labeled on the same day as the adhesion assay by loading with the fluorescent dye BCECF-AM (Molecular Probes, OR); 50 μg BCECF-AM was dissolved in 50 μl dimethyl sulfoxide (DMSO) and used to label 3×10⁷/mL of Molt-3 cells for 1 h. Cells were washed extensively with serum-free RPMI1640 to remove free label and resuspended in the same medium at 10⁶/mL. Labeled Molt-3 cells were added to peptide-treated monolayers and allowed to adhere for 45 min at 37° C. After three washes with HEPES/PBS, cells in each well were lysed with 0.5 mL of 2% Triton X-100 in PBS. Soluble lysates were transferred to 96-well (clear-bottom, black-sided) plates (Costar) for reading in a microplate fluorescence analyzer (Bio-Tek FL600) to give relative fluorescence (FL), i.e., the reading of fluorescence intensity of adherent corrected with the reading of cell monolayers only. Data were presented as the percentage of T-cell adherence calculated as follows:

Adherence (%)=(FL of treated samples/FL of control)×100 (1)

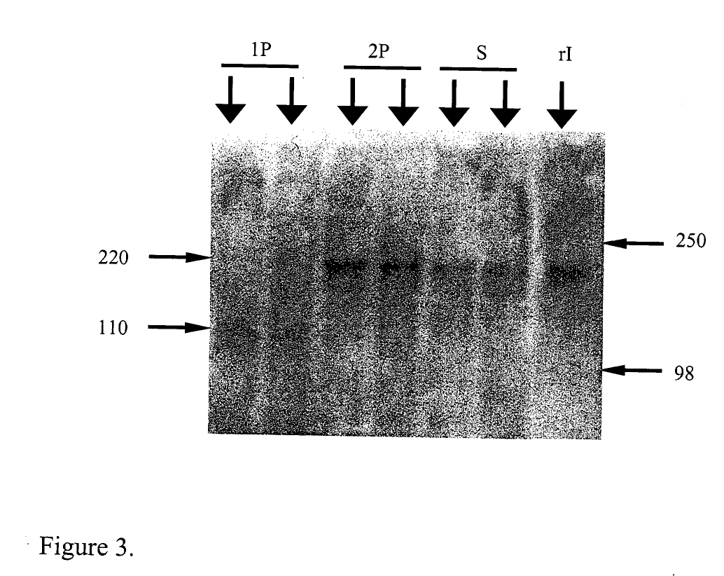

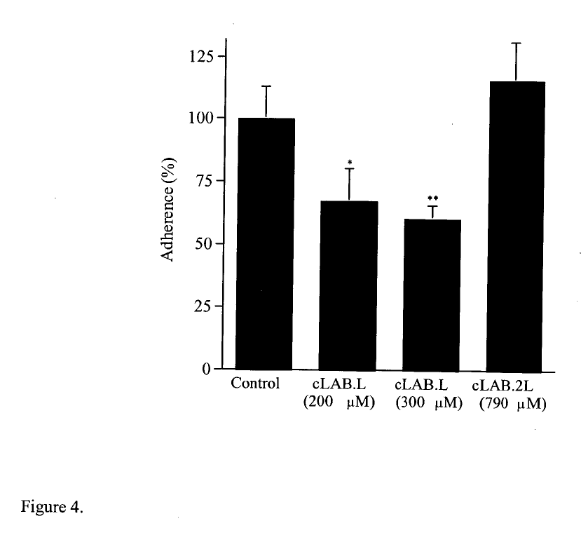

The results showed that Calu-3 cells express ICAM-1 that was resolved as both monomeric (˜110 kDa) and apparent dimeric (˜220 kDa) forms (FIG. 3). To mimic the inflammatory state of diseases where cytokines are released and ICAM-1 is upregulated, Calu-3 cells were induced for 48 h with 500 U/mL IFN-γ for the heterotypic adhesion experiment. The results indicate that the domain V peptide, cLAB.2L, did not interfere with the binding of BCECF labeled Molt-3 cells. On the contrary, the I-domain peptide, cLAB.L, inhibited this heterotypic cell adhesion by about 40% (FIG. 4). Indeed, the uptake study of the fluorescent-labeled cLAB.2L indicated that the cell binding of the peptide occurred with apparent Michaelis-Menten kinetics with no difference being observed between incubation at 4° C. and 37° C. (FIG. 3). Similar apparent binding constants were observed (Kd=56.5 μM and 47.82 μM; Bmax=20.16 and 24.27 fmole/μg protein, for 4° C. and 37° C. incubations, respectively).

### Example 6

DHFR Inhibition Assay

To measure DHFR inhibition by MTX and MTX-peptide conjugates (MTX-cIBR and MTX-cIBL), the rate of β-NADPH loss to form NADP was determined using a spectrophotometric assay. In this assay, one unit of DHFR was demonstrated to convert 1.0 μmol of 7,8-dihydrofolate and β-NADPH to 5,6,7,8-tetrahydrofolate and β-NADP per minute at pH 6.5 and 25° C. Into a 2.0 mL cuvette, a 1.0 mL solution of 0.11 mM β-NADPH and 33 μL of 2.3 mM DHFA were aliquoted and allowed to equilibrate. Then, a 10 μL aliquot of MTX or MTX-peptide conjugates was added to the reaction mixture at varying concentrations prepared by serial dilution of a stock solution. To begin the assay, 33 μL of DHFR (0.12-0.25 unit/mL with 0.1% BSA) was added to the reaction mixture. The absorbance of the reaction mixture was then recorded continuously for 5 min at 340 nm and the enzyme activity was determined by the rate of NADPH loss. Enzyme activity was determined for three different fixed concentrations of substrate (0.19, 1.9 and 10.0 mM DHFA). Sigmaplot v4.01 was then used to determine Kₘ and Vₘₐₓ values for MTX, MTX-cIBR and MTX-cIBL from Dixon plots of reciprocal enzyme activity (1/rate of NADPH loss) vs. inhibitor concentration, and the results are shown in Table 1.

TABLE I
Inhibition of DHFR Activity by MTX and MTX-conjugates

| Compound | Kₘ | Yₘₐₓ |
|---|---|---|
| MTX | 1.84 ± 0.7 × 10⁻⁹ M | 4.30 ± 0.42 × 10⁻⁴ |
| MTX-cIBR | 29.8 ± 2.1 × 10⁻⁹ M | 4.10 ± 0.18 × 10⁻⁴ |
| MTX-cIBL | 9.3 ± 0.7 × 10⁻⁹ M | 4.26 ± 0.07 × 10⁻⁴ |

The Kₘ value determined for MTX in this study was 1.84±0.7×10⁻⁹ M (Table I), which is similar to that found in the literature (Kₘ=2×10⁻⁹ M). In contrast, the Kₘ values of the MTX-cIBL and MTX-cIBR conjugates were approximately 4 and 15 fold times less than that of MTX, respectively. The fact that dihydrofolic acid, the natural substrate for DHFR, has only 1/10,000 of the affinity that MTX has for DHFR suggests that the MTX-conjugates would still be effective inhibitors of DHFR. The capacity (Vₘₐₓ) values for MTX and MTX-peptide conjugates were similar, indicating similar mechanisms of competitive inhibition of DHFR.

### Example 7

Cytotoxicity Assays

The cytotoxicity of MTX-peptide conjugates was evaluated in different cell lines, lincluding Molt-3 and L1210 T-cells, and KB epithelial cells. Cytotoxicity of Molt-3: MOLT-3 T-cells (2×10⁴ cells/mL) were incubated in a 96-well microtiter plate in the presence of different concentrations of either MTX or MTX-peptide conjugates in a final volume of 200 μL. After 72 h of growth, the relative numbers of viable cells were determined according to the manufacture's protocols for measuring cytotoxicity using a Dojindo Cytotoxicity Assay Cell counting Kit-8 (CCK-8). To measure the cell viability, 10 μL of CCK-8 was added to each well and the plate was incubated for 4 h. After incubation, the absorbance at 450 nm was measured using a UV plate reader. Cell growth in the presence of different drug concentrations was calculated relative to the value obtained in the absence of the drug. The IC₅₀ values were calculated using Sigmaplot v4.01, shown in Table 2. L1210 Mouse Leukemia Cell Growth Inhibition Assay: Exponentially growing L1210 (3×10⁵/mL) cells were diluted to 5×10⁴/mL in RPMI 1640 culture medium approximately 4 h prior to addition of MTX and MTX-peptide conjugates. Serial dilutions of compounds were prepared in un-supplemented medium, and 110 μL of drug solution was aliquoted in duplicate into 900 μL cell suspensions. Control flasks were treated with 110 μL of un-supplemented medium. Cells were incubated under standard conditions for 48 h, sufficient to allow control cells to divide approximately four times. Growth inhibition was determined by counting the cells on a Z2 model Coulter Counter (Coulter Electronics Ltd, Luton, Beds, UK). The IC₅₀ values were calculated using Graphpad Prism software (Graphpad Software, San Diego, Calif.). KB MTT Assay: KB cells were seeded in Falcons® 96 well plates (Becton-Dickinson Labware Europe, France) at a density of 1,500 cells/well in a volume of 0.18 mL culture medium and incubated under standard culture conditions for 24 h after seeding to allow entry into the exponential phase of cell growth. After this time, 20 μL of either MTX or MTX-peptide conjugates at appropriate dilutions were added to quadruplicate wells to give a final well volume of 200 μL. In each assay condition, control cells were treated with 20 μL of un-supplemented medium instead of drug. Cells were incubated for 96 h to allow control cells to divide approximately four times before cell viability was determined by MTT assay.

The number of viable cells after a 96-h incubation was determined by assessing their ability to reduce MTT (1-[4,5-dimethylthiazol-2-yl]-3,5-diphenylformazan) [21, 22]. Reduction occurs in the mitochondria, and thus, unlike other methods such as the sulforhodamine B assay, the MTT method can distinguish between viable and non-viable cells. A solution of 2 mg/mL solution (50 μL) of MTT (Sigma) in PBS was added to each medium-containing well and incubated for 1 h under standard culture conditions. After this time, the content of the wells were removed by inverting the plates over a sink and firmly blotting them on tissue paper to remove residual medium. The insoluble formazan crystals in each well were dissolved with 100 μL of DMSO by agitation on a shaker for 15 min. The absorbance of the solution in each well was measured at 540 nm on a MCC/340 model Titertak Multiscan® plate reader (Labsystems/Flow Laboratories, Oxfordshire, UK). The results were analyzed using Ascent Research software v.2.1 (Labsystems, UK).

TABLE II
IC₅₀ Values of MTX and MTX-peptide Conjugates in Various Cell Lines

| | IC50 in micromolar | | |
|---|---|---|---|
| Compound | Molt-3 T-cells | L1210 mouse leukemia cells | KB epithelial cells |
| MTX | 0.061 ± 0.02 | 0.014 ± 0.004 | 0.027 ± 0.006 |
| MTX-cIBR | 2.75 ± 0.8 | 5.5 ± 2.0 | NA* |
| MTX-cIBL | 2.68 ± 0.2 | 9.14 ± 1.0 | NA |
| MTX-VILPRG | 9.12 ± 0.2 | 0.7 ± 0.3 | NA |
| MTX-PRGGSV | 5.13 ± 0.6 | 1.6 ± 0.4 | NA |

*NA = no activity at 10 μM.

Comparing the IC₅₀ values from Table II within each T-cell line, it is apparent that MTX-peptide conjugates are less toxic than MTX. On the other hand, the conjugates were toxic to the LFA-1-expressing cell lines (Molt-3 and L1210) but not to KB epithelial cells. In Molt-3 T-cells, the cyclic peptide conjugates (MTX-cIBL and MTX-cIBR) were more toxic than the linear peptide conjugates: MTX-VILPRG and MTX-PRGGSV. However, the linear peptide conjugates are more toxic than the cyclic peptide conjugates in L1210 cells. The difference in selectivity of the cyclic and linear conjugates may be due to the recognition of these peptides by LFA-1 expressed on human Molt-3 and mouse L1210 T-cells. Finally, in the KB epithelial cell line, MTX had an IC₅₀ of 0.027 μM. In contrast, the MTX-conjugates had no activity at concentrations up to 10 μM. The inactivity of MTX-peptide conjugates is likely due to the inability of KB cells to internalize these conjugates because the cells do not express LFA-1 receptors. These results demonstrate the importance of the LFA-1 receptor for the internalization and activity of the MTX-conjugates, where the peptide is derived from ICAM-1 protein.

Peptides Derived from LFA-1.

We determined whether the treatment of peptides, MTX, and MTX-peptide(s) on HCAEC and Molt-3 cells result in the inhibition of cell proliferation. Cell viability was assessed by propidium iodide (PI) assay for double stranded polynucleic acids (PNA). HCAEC cells were plated in a volume of 100 μl/well in 96-well cell culture plate using GIBCO non-CO₂-buffered culture medium (Life Technologies, Gaithersburg, Md.) with 5% fetal calf serum and 2 mM L-glutamine (Sigma). The method applies the 1+2 day screening protocol of U.S. National Cancer Institute (NCI) in which cells are allowed to recover for 1 day from the trauma of dissociation during seeding, then incubated with test compounds for an additional 2 days. At the end of incubation, plates were harvested by freezing at −30° C. for at least 2 h and thawed at 50° C. for 15 min. 40 μg/mL of PI was added to each well, followed by incubation in the dark for 60 min at room temperature. The PI fluorescence was read using microplate fluorescence analyzer (Bio-Tek FL600) at 530-nm excitation and 620-nm emission at which PI fluorescence is independent of culture protein. The effect of the test compound was calculated by taking into account the fluorescence of blanks (cell, medium and compound solution) at the time zero and at the end of incubation period. The qualitative effect of the compounds on cell growth and cytotoxicity based on the relative amount of the remaining cellular PNA can be graded as causing: growth stimulation, partial growth inhibition, total growth inhibition, net cell killing, and total culture extinction.

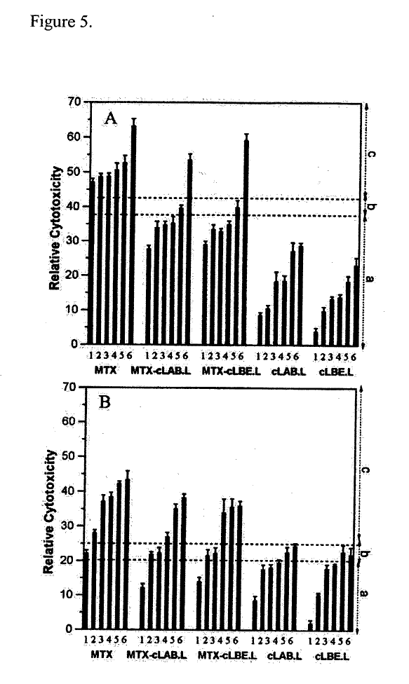

The results indicate that the effects of the test compounds on both HCAEC and Molt-3 T-cells falls within the category of partial inhibition, total growth inhibition, or net cell killing (FIGS. 5A and 5B), neither growth stimulation nor total culture extinction was observed in this study. Treatment with MTX causes net cell killing in HCAEC at all the test concentrations (FIG. 5A), this effect was found at ≧1.0 μM in Molt-3 T-cells (FIG. 5B). The MTX-peptide(s) appear to be less toxic than the free MTX; while the net cell killing due to MTX treatment occurred in HCAEC at ≧0.1 μM, the same effect due to MTX-peptide(s) only emerged at ≧500 μM (FIG. 5A). In Molt-3 cells, net cell killings were observed at ≧1.0 and ≧50 μM for MTX and MTX-peptide(s), respectively (FIG. 5B). Free peptides exhibit a relatively low toxicity in both cells. All the test concentrations only result in partial growth inhibition in HCAEC (FIG. 5A). Meanwhile, a total growth inhibition by cLAB.L and cLBE.L was emerged in Molt-3 cells at 100 μM (FIG. 5B). However, a five-fold increase in peptides concentration to 500 μM did not elevate the effect to total cell killing of Molt-3 cells.

### Example 8

MTX-cIBR Internalization by LFA-1 Receptor

To study the involvement of LFA-1 in the internalization of MTX-peptide conjugates, MTX-cIBR toxicity was evaluated in Molt-3 T-cells in the presence of increasing concentrations of cIBR peptide (10, 100, 1000 □M) or an anti-LFA-1 antibody (clone 38) at 40 and 80 μL/mL. Molt-3 T-cells (2×10⁴ cells/mL) were incubated in a 96-well microtiter plate in the presence of either cIBR peptide or an anti-LFA-1 antibody (clone 38) at various concentrations. As a control, some cells were left untreated. The MTX-cIBR conjugate was then added to each well to a final concentration of 1 μM. As a reference for minimal metabolic activity, 10 mM of the succinate dehydrogenase inhibitor iodoacetamide (IAA) was added to untreated wells. After 72 h of continuous exposure, the relative number of viable cells was determined using an MTT assay, except after 4 h of incubation with a 5.0 mg/mL solution of MTT, the content of each well was transferred to a microcentrifuge tube. The tubes were spun to pellet the cells and the supernatant was carefully removed. The formazan crystals were dissolved in 200 μL of 0.04 N HCl in isopropanol; the tubes were sonicated for 5 min to completely dissolve crystals and then re-centrifuged to pelletize the cell debris. 100 μL aliquots of the supernatant solutions were removed and transferred to a 96-well microtiter plate. The optical density of the solution was measured at 570 nm using a UV plate reader. The measured cell metabolic activity is given in Table 3.

TABLE 3
Protective Effect of cIBR Peptide or CD11a Antibody
against MTX-cIBR Activity (1 μM).

| cIBR | | | |
|---|---|---|---|
| peptide [μM] | % viable cells | CD11a antibody dose | % viable cells |
| 10 | 58 ± 3 | 40 μL/mL | 35 ± 7 |
| 100 | 81 ± 7 | 80 μL/mL | 97 ± 10 |
| 1000 | 94 ± 5 | | |

This result suggests that the internalization of MTX-cIBR is mediated by the LFA-1 receptor, and that the peptide fragment of MTX-cIBR binds to the I-domain of LFA-1. In a similar experiment, the cIBR peptide reduced the cytotoxicity of MTX-cIBR in a concentration dependent manner, indicating that peptide conjugation to MTX did not alter its binding properties.

### Example 9

Effect of MTX-Conjugation on cIBR Peptide Binding to LFA-1

The binding of MTX-peptide conjugates to LFA-1 in response to LFA-1 activation was evaluated. As necessary, cells were activated with 10% v/v phorbol 12-myristate-13-acetate (PMA) containing medium to a final concentration of 2 μM PMA and incubated for 16 h. 200 μL aliquots of Molt-3 T-cells (1×10⁶ cells/mL in PBS/BSA 1%) were added to 48-well plates and treated with either cIBR peptide or MTX-cIBR conjugate at concentrations of 1, 10, or 100 μM for 45 min at 4° C. The cells were then washed to remove unbound cIBR or MTX-cIBR. T-cells were centrifuged for 3 min at 1800 rpm, the supernatant was decanted by flicking off excess liquid, and the cells were re-suspended in 500 μL of PBS. The cells were then re-centrifuged, supernatant was removed, and cells were re-suspended again in 150 μL of PBS/BSA 1%. Next, 50 μL of an FITC-labeled anti-CD11a antibody (clone 38, 10 μg/L) was added and incubated for 45 min at 4° C. followed by washing. After the 45 min of incubation with FITC-labeled antibody, cell samples were transferred to Eppendorf tubes and centrifuged at 3000 g for 3 min. The supernatant was decanted, and the pellet was washed twice with 10 mM HEPES/PBS. The cells were then fixed with ice-cold 2% w/v paraformaldehyde/PBS for 20 min. Samples were analyzed using a Becton-Dickinson FACScan flow cytometer with 3.2.1 fl software for data analysis and acquisition. Reduction in binding of the FITC-labeled antibody was calculated as a fraction of fluorescence remaining after incubation with cIBR or MTX-cIBR compared to the fluorescence of FITC-antibody binding untreated cells. The results showed that the conjugation of MTX does not interfere with the binding of the cIBR peptide fragment in the MTX-cIBR conjugate to the LFA-1 receptor, and the binding of the MTX-cIBR was specific to the LFA-1 receptor.

### Example 10

Effect of MTX and MTX-Conjugates on Cell Cycle

Cell cycle analysis was performed in order to evaluate the effect of MTX-peptide conjugation on the ability of MTX to inhibit DNA synthesis and arrest cell cycle. After 3, 6, 9, 12, 24, 36 and 48 h of incubation with MTX, MTX-cIBR or MTX-PRGGSV at a concentration of 1 μM, L1210-1565 cells were harvested by centrifugation at 450×g (2500 rpm) for 5 min at room temperature. The cell pellets were resuspended and fixed in 2.5 mL of ice-cold 70% ethanol. These pellets were stored at 4° C. before analysis using flow cytometry.

PI staining of fixed cell pellets for cell cycle analysis: One day prior to analysis, cells were centrifuged at 450×g (2500 rpm) for 5 min at room temperature and the pellets re-suspended in 0.8 mL PBS followed by addition of 0.1 mL each of 1 mg/mL ribonuclease A (RNase A; Sigma) and 0.4 mg/mL PI (Sigma). RNA digestion by RNase A is required to avoid the intercalation of PI into the double-stranded regions of this nucleic acid, which interfers with the measurement of DNA. After a 30 min incubation at 37° C., samples were wrapped in aluminum foil and stored at 4° C. overnight. Cell cycle analysis was performed using a Coulter EPICS Elite ESP (Beckman Coulter, Buckinghamshire, UK) equipped with an argon-ion laser tuned to 488 nm and red fluorescence collected at 630 nm. DNA histograms were produced and analyzed using the Winmidi software package (v.2.8, written by J.Trotter, University of Cardiff, UK).

The results indicate that at times up to 12 h, MTX and the MTX-peptide conjugates did not demonstrate a shift in the DNA histogram compared to an untreated control group. However, after 24 h, cells treated with MTX demonstrated a significant increase in the S phase population and a depletion of cells in G₂ phase, signifying the arrest of cell cycle. At 24 h, MTX-PRGGSV also caused an increase in the S phase population; however, the distribution was not entirely similar to MTX with most of the cells arrested earlier in the S phase compared to MTX. After 36 h of treatment MTX-cIBR also demonstrated the arrest of cells in the S phase with a histogram similar to that of MTX-PRGGSV. The results indicate that the uptake of the MTX-peptide conjugate is mediated primarity by LFA-1, and the delay in the arrest of cell cycle by MTX-cIBR compared to MTX-PRGGSV may be due to factors such as peptide size, metabolism, internalization kinetics, or affinity for DHFR.

### Example 11

Thymidine Synthase (TS) Inhibition Studies

The ability of MTX-conjugates and MTX to inhibit TS was evaluated in continuous exposure assays and wash-out studies.

Continuous Exposure Studies: The cell line L1210-1565 was used to study the ability of MTX and MTX-peptide conjugates to inhibit TS using a whole cell assay. Cell suspensions of 5 mL at 1×10⁵ cells/mL were treated with either MTX or MTX-peptide conjugates at 3 μM continuously for 4 h. An equivalent amount of unsupplemented medium was added to control flasks. On the day of each experiment, a fresh solution of unlabelled deoxyuridine (dUrd) was prepared in deionized H₂O (dH₂O) and was added to a stock solution of [5-³H]-dUrd (22 Ci/mmole) to give a concentration of 300 μM and a specific activity of 3.3 Ci/mmole (˜7260 dpm/pmole).

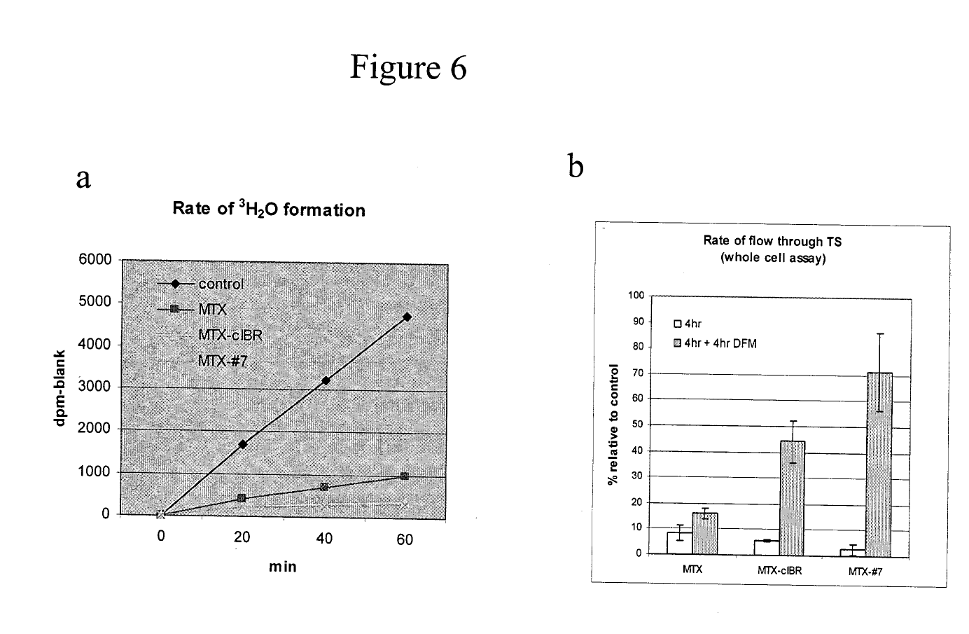

To begin the assay, the 300 μM [5-³H]-dUrd stock solution was diluted ten fold in deionized H₂O and 50 μL of this solution was added to each culture flask to give a final concentration of 0.03 μM. The rate of ³H₂O formation was measured over a 1 h period (20, 40 and 60 min) by removing a 3×0.4 mL aliquot of the cells in culture medium and mixing it with 0.4 mL of ice-cold 1.0 M perchloric acid (PCA; Sigma) in microfuge tubes. Then, 0.5 mL of an ice-cold charcoal suspension containing 200 mg/mL activated charcoal (Sigma) and 10 mg/mL dextran (Sigma) in deionized H₂O was added to the microfuge tubes and incubated at 4° C. for 15 min. After this time, the microfuge tubes were centrifuged at 13,000 rpm for 4 min at room temperature (MSE Micro-Centaur microfuge, Sanyo Gallenkamp PLC, Crawley, Sussex, UK) and 0.5 mL of the ³H₂O-containing supernatant was mixed with 10 mL of Ultima Gold scintillation fluid in 20 mL polyethylene scintillation vials (Canberra Packard, Pangbourne, Berkshire, UK). For each time point, radioactivity was determined by counting each vial on the tritium channel of a Tri-Carb 2000CA Model Liquid Scintillation Analyzer (Canberra Packard, Pangbourne, Berkshire, UK). Background radioactivity was assessed by cooling a flask of untreated cells on ice before the start of each experiment and adding 50 mL of the [5-³H]-dUrd solution. Aliquots of 3×0.4 mL aliquots of cells in culture medium were added to 1.0 M PCA-containing microfuge tubes and the rate of ³H₂O release was analyzed as described above. The rate of 3H₂O formation using background-corrected samples was calculated by fitting the data to a linear regression model using Fox85 software (v.6, written by L. Hart, ICR). The slope represents the amount of ³H₂O formed in dpm/min, which is standardized to pmoles of ³H₂O released/min/10⁶ cells. The results are given in FIG. 6a. After a 4 h incubation with MTX, MTX-cIBR, or MTX-PRGGSV, the production of ³H₂O was inhibited to a similar degree, suggesting that the conjugates are also effective inhibitors of the TS enzyme.

Washout studies: To describe the efflux of TS inhibitors and consequent relief of TS inhibition, the same method described above was used. However, after cell lines were treated with MTX or MTX-peptide conjugates for 4 h, the cells were pelleted and resuspended in fresh medium without drug. Cells were then incubated for an additional 4 h before TS activity was determined. The results are given in FIG. 6b. FIG. 6b shows that MTX maintained its ability to inhibit TS, which confirms previous studies demonstrating that polyglutamated MTX is retained within cells as a “drug-depot”. However, the ability of these MTX-conjugates to inhibit TS activity after 4 h incubation in DFM was less than that of MTX. Interestingly, the cyclic peptide conjugate (MTX-cIBR) retained more activity than the linear conjugate, MTX-PRGGSV. This may suggest that linear MTX-peptides are more susceptible to enzymatic metabolism during 4 h DFM incubation than the cyclic conjugates, which may affect the ability of these conjugates to be retained and continue to inhibit TS.

### Example 12

TNF-α Assay

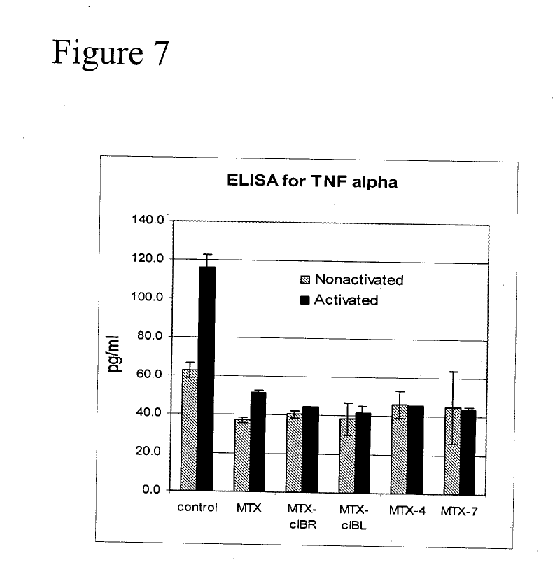

An ELISA assay was used to evaluate the ability of MTX-peptide conjugates to inhibit the production of TNF-α compared to MTX alone in resting and stimulated human peripheral blood leukocytes (PBL). Human PBL were isolated as described previously and TNF-α production was induced in the following manner. PBL, 1×10⁷ cells/mL, were aliquoted into wells of a 96-well plate and activated with PMA and ionomycin at final concentrations of 0.2 μg/mL and 10 μM respectively. As a control, some cells were not activated to demonstrate a background TNF-α level in culture. Then, both non-activated and activated cells were treated with either MTX or MTX-peptide conjugate to give a final concentration of 10 nM. After 48 h of incubation, 100 μL of culture supernatant was removed from each well and assayed for cytokine concentration. A human TNF-α ELISA kit (eBioscience, cat. 88-7346) was used to quantify TNF-α produced by human PBMC in vitro. The results are given in FIG. 7, and show that the MTX-peptide conjugates are as effective as MTX alone in suppressing TNF-α production, suggesting that MTX conjugation to ICAM-1 peptides does not affect the ability of MTX to suppress TNF-α production.

### Example 13

Modulation of Inflammatory Cytokine Production by LFA-1 Peptides, MTX, and MTX-Peptide Conjugates

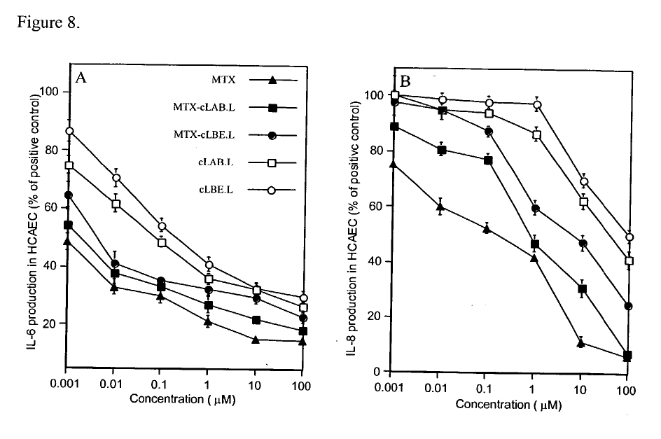

It is well known that some anti-inflammatory agents can modulate the secretion of inflammatory cytokines. Thus, to test whether the LFA-1 peptides, MTX and MTX-peptide(s) are able to suppress the production of IL-6 and IL-8, HCAEC cell monolayers were stimulated with TNF-α, as a known physiological stimulus of endothelial inflammation, in the presence of the test compounds. The effects of these compounds (0.001-100 μM) on the IL-6 and IL-8 productions in HCAEC are demonstrated in FIG. 8. MTX and MTX-peptide(s) are better inhibitors of IL-6 and IL-8 production than the free peptides. MTX and MTX-peptide(s) partly block the production of IL-6 with relatively similar potency. On the other hand, to block the IL-6 production by >50%, the free peptides require approximately a 100 fold concentration compared to that of MTX and MTX-peptide(s) (FIG. 8A). MTX-peptide(s) only begins to effectively reduce the IL-8 production at ≧0.1 μM. At ≦1 μM, neither cLAB.L nor cLBE.L affected the IL-8 production (FIG. 8B). Overall, the peptide conjugation decreases the efficacy of MTX in inhibiting the cytokine production; the effect is more pronounced in the IL-6 than in IL-8 production.

### Example 14

In vivo Activity of MTX-cIBR Conjugate

The in vivo activity of MTX-cIBR was compared to MTX alone in collagen induced rheumatoid arthritis (CIA) animal model. In this study, the treatment of mice with MTX-cIBR conjugate was done after the mice had arthritis at 5 weeks time period with the average arthritis score of two. The MTX-cIBR conjugate was injected intravenously via the tail vein as a bolus dose in aqueous solution (100 mg per mouse) once daily, after day 35 for either one, three or five days. The positive control received intravenous injections of saline while another treatment group received MTX injections (molar equivalent dosage to conjugate) for five days. Clinical arthritis symptoms were evaluated weekly for up to sixteen weeks. Arthritis development was evaluated by several parameters, including the percent of animals acquiring arthritis, arthritis index (AI) score of limbs, changes in paw-volumes and histologic score for the joint.

Mice injected with collagen adjuvant developed pronounced inflammation of the joints, as evidenced by swelling and erythema. In contrast, the treatment with the conjugate resulted in the arthritis index score for the limb decreasing from two at week 8 to zero at week 12. Joint damage following treatment with the conjugate and MTX was compared to the control group following histopathologic examination of the joints for signs of inflammation, fibrillation cartilage destruction, ebumation, pannus and bone degeneration. Total joint damage (TJD) was calculated as the sum the scores for each limb assigned by the pathologist on a scale of 0 to 3 (where 0 is no change and 3 is gross histological change). TJD in the control group was 8.4±2.8 and 7.4±2.5 for MTX treatment compared to 4.4±2.1 for the MTX-cIBR conjugate treated group. In the MTX-cIBR conjugate group only 10% of the mice showed signs of joint eburnation (bone-on-bone resulting from cartilage degeneration), compared to 44% in MTX treated mice and 50% in the control group. Thus, treatment of mice with the MTX-cIBR conjugate shows a trend towards less joint damage, and the conjugate effectively stops the progression of rheumatoid arthritis.

### Example 15

Preparation of Tablets

The MTX-cIBR conjugate (10.0 g) is mixed with lactose (85.5 g), hydroxypropyl cellulose HPC-SL (2.0 g), hydroxypropyl cellulose L-HPC, LH-22 (2.0 g) and purified water (9.0 g), the resulting mixture is subjected to granulation, drying and grading, and the thus obtained granules are mixed with magnesium stearate (0.5 g) and subjected to tablet making, thereby obtaining tablets containing 10 mg per tablet of the MTX-cIBR conjugate.

### Example 16

Administering to a Subject

A subject suffering from rheumatoid arthritis is identified. The tablet prepared in Example 15 is provided to the subject at time 0, and one tablet every 24 h for a period of 6 months is given. After administration of the last tablet, the condition of the subject is reevaluated. The treated subject exhibits symptoms of RA that are less severe compared to the subject that was not treated.

All printed patents and publications referred to in this application are hereby incorporated herein in their entirety by this reference.

While the preferred embodiment of the invention has been illustrated and described, it will be appreciated that various changes can be made therein without departing from the spirit and scope of the invention.

We claim:

1. A compound of formula P-L-M wherein:
P is a peptide comprising 4 to 12 contiguous amino acid residues from an ICAM-1 or an LFA-1 protein sequence;
L is a direct bond or a linker having from 1 to 20 carbon atoms; and
M is a reporter molecule, a dye, or a drug.

2. The compound of claim 1, wherein the peptide is a linear peptide.

3. The compound of claim 2, wherein the peptide further comprises Xaa and Cys as terminal amino acids, wherein Xaa is Pen or Cys.

4. The compound of claim 3, wherein the peptide is cyclized.

5. The compound of claim 1, wherein the peptide is cyclic.

6. The compound of claim 1, wherein the peptide is from the LFA-1 protein sequence.

7. The compound of claim 6, wherein the peptide is selected from the insert (I) domain, the cation binding domain V and VI, or the I-domain like region of LFA-1.

8. The compound of claim 7, wherein the peptide is selected from the group consisting of SEQ ID Nos: 4-23, 25, and 60.

9. The compound of claim 7, wherein the peptide is selected from the group consisting of 4-7, 10, 14-17, 20, and 60.

10. The compound of claim 1, wherein the peptide is from an ICAM-1 protein sequence.

11. The compound of claim 10, wherein the peptide is selected from the D1 region of ICAM-1.

12. The compound of claim 11, wherein the peptide is selected from the group consisting of SEQ ID Nos: 28-54.

13. The compound of claim 11, wherein the peptide is selected from the group consisting of SEQ ID Nos: 28-30.

14. The compound of claim 11, wherein the peptide is selected from the group consisting of SEQ ID Nos: 31-33.

15. The compound of claim 10, wherein an amino acid residue of the peptide is an unnatural amino acid or an analogue amino acid.

16. The compound of claim 15, wherein the unnatural amino acid comprises the D-isomer.

17. The compound of claim 15, wherein the analogue amino acid is a lysine.

18. The compound of claim 17, wherein the peptide is selected from the group consisting of SEQ ID Nos: 39-46.

19. The compound of claim 15, wherein the analogue amino acid comprises C-terminal amide.

20. The compound of claim 1, wherein the linker is a direct bond.

21. The compound of claim 1, wherein the linker comprises 4 amino acid residues.

22. The compound of claim 1, wherein the drug is selected from the group consisiting of methotrexate, lovastatin, taxol, ajmalicine, vinblastine, vincristine, cyclophosphamide, fluorouracil, idarubicin, ifosfamide, irinotecan, 6-mercaptopurine, metomycins, mitoxantrone, paclitaxel, pentostatin, plicamycin, topotecan, fludarabine, etoposide, doxorubicin, doxetaxel, danorubicin, albuterol, and propidium.

23. The compound of claim 22, wherein the drug is methotrexate.

24. The compound of claim 22, wherein the drug is fluorouracil.

25. A compound of formula cPRGX_{bb}SK or cPRX_{bb}GSK, where X_{bb} is a neutral, hydrophobic or charged residue selected from the group consisting of N, F, V, D, and R.

26. The compound of claim 25, wherein X_{bb} is N.

27. A compound of formula:
```
Pro-Arg-Gly
| |
Lys-Ser-Xbb
  |
  L
  |
  M
```
wherein X_{bb} is a neutral, hydrophobic or charged residue selected from the group consisting of
Asn, Phe, Val, Asp, and Arg;
L is a direct bond or a linker having from 1 to 20 carbon atoms; and
M is a reporter molecule, a dye, or a drug.

28. The compound of claim 27, wherein X_{bb} is Asn or Asp.

29. The compound of claim 27, wherein L is a direct bond.

30. The compound of claim 27, wherein L is a linker comprising 4 amino acid residues.

31. The compound of claim 27, wherein M is methotrexate.

32. The compound of claim 27, wherein M is taxol.

33. A pharmaceutical composition comprising a therapeutically effective amount of a compound of claim 27 in admixture with a pharmaceutically acceptable carrier.

34. A method of treating a patient comprising administering to a patient a therapeutically effective amount of a compound of claim 27.

35. The method of claim 34, wherein the patient has a disease selected from the group consisting of cancer, rheumatoid arthritis, multiple sclerosis, lupus, and HIV.

36. A method of treating a subject, the method comprising administering a therapeutically effective amount of a compound of formula P-L-M wherein:
P is a peptide comprising 4 to 12 contiguous amino acid residues derived from ICAM-1 or LFA-1 protein sequence;
L is a direct bond or a linker having from 1 to 20 carbon atoms; and
M is a reporter molecule, a dye, or a drug in admixture with a pharmaceutically acceptable carrier.

37. The method of claim 36, wherein the drug is selected from the group consisiting of methotrexate, lovastatin, taxol, ajmalicine, vinblastine, vincristine, cyclophosphamide, fluorouracil, idarubicin, ifosfamide, irinotecan, 6-mercaptopurine, metomycins, mitoxantrone, paclitaxel, pentostatin, plicamycin, topotecan, fludarabine, etoposide, doxorubicin, doxetaxel, danorubicin, albuterol, and propidium.

38. The method of claim 37, wherein the drug is methotrexate.

39. The method of claim 36, wherein the subject is a mammal.

40. The method of claim 39, wherein the mammal is human.
